(19) United States
(12) Patent Application Publication
(10) Pub. No.: US 2024/0277641 A1
(43) Pub. Date: Aug. 22, 2024

(54) METHODS FOR TREATING NERVOUS SYSTEM DISORDERS WITH ANTIPURINERGIC AGENTS

(71) Applicant: PAXMEDICA, INC., TARRYTOWN, NY (US)

(72) Inventors: Zachary Rome, Nyack, NY (US); Michael Derby, Greenwich, CT (US); David W. Hough, Yardley, PA (US)

(21) Appl. No.: 18/435,069

(22) Filed: Feb. 7, 2024

Related U.S. Application Data
(63) Continuation of application No. PCT/US2022/041050, filed on Aug. 22, 2022.
(60) Provisional application No. 63/236,155, filed on Aug. 23, 2021.

(51) Int. Cl. A61K 31/17; A61K 9/00; A61K 47/10; A61K 47/14; A61K 47/40; A61K 47/44

(52) U.S. Cl. CPC A61K 31/17; A61K 9/0019; A61K 9/0043; A61K 9/0053; A61K 47/10; A61K 47/14; A61K 47/40; A61K 47/44

(57) ABSTRACT

The present invention provides compositions and methods for treating nervous system disorders in a mammal. These compositions and methods comprise administering an effective amount of an antipurinergic agent according to a pharmacokinetic and/or pharmacodynamic method comprising an optional loading dosing regimen and a subsequent maintenance dosing regimen, to achieve efficacy in view of a dynamic, nonlinear correlation between efficacy and blood levels of the agent.

### CROSS-REFERENCE TO RELATED APPLICATION

This application is a continuation of International Application Number PCT/US2022/041050 filed Aug. 22, 2022, which claims priority to U.S. Provisional Application No. 63/236,155 filed Aug. 23, 2021, the entirety of which is incorporated by reference herein.

### FIELD OF THE INVENTION

The present invention provides compositions and methods for treating nervous system disorders in a mammal, and are particularly useful for maximizing therapeutic efficacy while minimizing undesirable side effects. These compositions and methods comprise administering an effective amount of an antipurinergic agent according to a pharmacokinetic and/or pharmacodynamic dosing regimen. This dosing regimen comprises an optional loading dosing regimen and a subsequent maintenance dosing regimen to achieve optimal blood levels in view of a heretofore unknown dynamic, nonlinear correlation between efficacy and blood levels of the agent over time. Each loading dose of the loading dosing regimen contains from about 3 mg/kg to about 30 mg/kg of the antipurinergic agent and is administered as a single dose or as multiple doses each administered with a frequency ranging from about once daily to about once every third month. The maintenance doses of the maintenance dosing regimen each contain from about 1 mg/kg to about 15 mg/kg of the antipurinergic agent and are administered with a frequency ranging from about three times daily to about once every third month. Because of the nonlinear correlation between efficacy and blood levels, the dosing regimen and dosage levels of the present invention would not have been predicted based on previously disclosed dose-response linearity.

### BACKGROUND OF THE INVENTION

Nervous system disorders, whether mild or severe in their manifestation, affect many individuals in the US and around the world. These disorders have an impact beyond the individual patient and affect family members, caregivers, and society in general.

Nervous system disorders, include, cognitive, social, or behavioral disabilities, nervous system and neurodevelopmental disorders, psychiatric disorders, neurologic disorders, and central nervous system (CNS) disorders. These nervous system disorders include, inter alia, autism spectrum disorder (ASD), fragile X syndrome (FXS), fragile X-associated tremor/ataxia syndrome (FXTAS), myalgic encephalomyelitis/chronic fatigue syndrome (ME/CFS), post-traumatic stress syndrome (PTSD), Tourette's syndrome (TS), Parkinson's Disease, Angelman Syndrome (AS), and the CNS disorder manifestations often associated with Lyme disease and other tick-borne diseases, and the nervous system and central nervous system (CNS) disorders associated with COVID-19 and other viruses (e.g. Epstein Barr Human Herpesvirus 6 and 7, Herpes Simplex Virus, Cytomegalovirus, and others), including their long term effects. Note that this list of nervous system disorders is exemplary and that there are many others which can benefit from the present invention.

Current treatments for these exemplified disorders are limited and often targeted to specific symptoms such as seizures, anxiety, depression, attention deficit/hyperactivity, sleep disorders, cognitive impairment, and the like. Even though there is much research in the area and the potential for new or known therapeutic agents for such treatments, it is not always apparent how to safely and effectively dose these agents. It is demonstrated herein as set forth in the examples, that antipurinergic agents can be administered for treating these disorders according to a pharmacokinetic and pharmacodynamic treatment regimen that would not have been predicted a priori. These agents were administered at dosages and frequencies not previously disclosed or contemplated in the scientific literature, which led to the discovery of a dynamic, nonlinear correlation between efficacy and blood levels of the agent over time.

Autism is associated with a combination of genetic and environmental factors and has been reported to have an incidence in the US of about 1 in 60 children. Global prevalence estimates for autism are about 25 million individuals. Autism is also referred to as autism spectrum disorder (ASD), because it includes a broad range of symptoms characterized by challenges with social skills, repetitive behaviors, speech, and nonverbal communication. In 2013, the American Psychiatric Association merged four distinct autism diagnoses into the single diagnosis of autism spectrum disorder. This single diagnosis includes autistic disorder, childhood disintegrative disorder, pervasive developmental disorder-not otherwise specified (PDD-NOS), and Asperger syndrome. Signs and symptoms of autism usually appear by age 2 or 3. Autism spectrum disorder is a condition related to brain development that impacts how a person perceives and socializes with others, and can cause problems with social interaction and communication. The disorder can also include limited and repetitive patterns of behavior.

Research shows that early intervention of autism spectrum disorder can lead to positive outcomes as described in the following references: Chaste P, Leboyer M (2012). “Autism risk factors: genes, environment, and gene-environment interactions”. Dialogues in Clinical Neuroscience. 14 (3): 281-92. PMC 3513682. PMID 23226953; and Centers for Disease Control and Prevention Morbidity and Mortality Weekly Report, Prevalence of Autism Spectrum Disorder Among Children Aged 8 Years Autism and Developmental Disabilities Monitoring Network, 11 Sites, United States, 2014 Surveillance Summaries/Apr. 27, 2018/67(6); 1-23.

There is currently no cure for autism spectrum disorder, and no US FDA approved medications to treat the core symptoms. According to the American Psychiatric Association's (APA's) Diagnostic and Statistical Manual of Mental Disorders (DSM-V) diagnostic criteria, the core symptoms of Autism Spectrum Disorder include: persistent deficits in social-emotional reciprocity which results in difficulty developing, maintaining, and understanding relationships; deficits in verbal and nonverbal social communication; and restricted, repetitive patterns of behavior, interests or activities Persons with ASD often have many associated (i.e. non-core) symptoms including hyper- or hypo-reactivity to sensory input or unusual interest in sensory aspects of the environment, clinically significant impairment in social, occupational, or other important areas of current functioning, cognitive impairment, impulsiveness, attention deficit and hyperactivity symptoms, sleep disturbances, gastrointestinal complaints and food/chemical sensitivities, unusual eating habits, depression, mood disorders, anxiety, seizures, irritability, temper outbursts, sometimes violent behavior which can be self-directed or directed towards others.

Despite the prevalence of these core symptoms, instead, the focus of current therapies is on treating some of the accompanying non-core symptoms with various medications such as antipsychotics, anxiolytics, antidepressants, stimulants or medications for insomnia. Non-core symptoms that are often manifested include depression, seizures, anxiety, sleep disorders, hyperactivity, and trouble focusing. Also, behavioral, occupational, and speech therapies and other non-pharmacological interventions are employed. However, the exact causes of autism are not fully understood, thus contributing to the challenges of new drug development program.

Fragile X syndrome (FXS) is a rare, genetic neurodevelopmental disorder that affects approximately 1 in 4,000 people in the US. It is associated with highly variable cognitive and behavioral manifestations and has many overlapping features with ASD. The syndrome is an X-linked disorder, meaning that the genetic mutation occurs on the X chromosome. In FXS, there is a trinucleotide repeat expansion in the FMR1 gene. A trinucleotide expansion is a particular gene mutation in which a sequence of three nucleotide base pairs improperly repeats itself multiple times. In the case of FXS, the repeating trinucleotide sequence is cytosine-guanine-guanine (CGG). Normally, this DNA segment is repeated from 5 to about 40 times. In people with FXS, the segment is repeated more than 200 times. This excessive repetition typically results in no functional FMR1 mRNA transcript being produced, and the protein that is normally encoded by this transcript—fragile X mental retardation protein (FMRP)—is also absent.

Fragile X-associated tremor/Ataxia (FXTAS) is a different disorder than FXS, but genetically related to FXS. It is an “adult onset” rare, genetic neurodegenerative disorder, usually affecting males over 50 years of age. Females comprise only a small part of the FXTAS population, and their symptoms tend to be less severe. FXTAS affects the neurologic system and progresses at varying rates in different individuals.

FXS patients have the “full mutation” in the FMR1 gene (typically well over 200 CGG trinucleotide repeats), but patients with FXTAS are considered premutation ‘carriers’ of the FMR1 gene, as they have CGG trinucleotide repeats numbering in the range of 55-200. The function of the FMR1 gene is to make a protein (FMRP) that is important in brain development and for the maintenance and regulation of synaptic connections between neurons. Researchers believe that (for unknown reasons) having the premutation leads to the overproduction of FMR1 mRNA (which contains the expanded repeats). Researchers also suspect that the high levels of mRNA are what cause the signs and symptoms of FXTAS, but more research is needed to confirm these hypotheses.

Individuals with FXTAS usually experience symptoms after the age of 55. As premutation carriers age, especially men, the likelihood of experiencing symptoms rises. This likelihood reaches 75 percent by age 75 for premutation men. The progression of symptoms, including memory loss, slowed speech, tremors, and a shuffling gait, is gradual, with interference in daily activities by tremors and falls occurring around ten years after onset of the first symptoms. Dependence on a cane or walker occurs approximately 15 years after first exhibiting the symptoms of the disorder. Some people with FXTAS show a step-wise progression (i.e., symptoms plateau for a period of time but then suddenly worsen) with acute illnesses, major surgery, or other major life stressors causing symptoms to worsen more quickly.

The prevalence of FXTAS is unknown, although current estimates suggest that about 30%-40% of male FMR1 premutation carriers over 50 years of age, within families already known to have someone with Fragile X, will ultimately exhibit some features of FXTAS. There is no FDA approved therapy for FXTAS and currently used treatments only address the symptoms of the condition, rather than targeting the pathophysiology itself.

Myalgic encephalomyelitis/chronic fatigue syndrome (ME/CFS) can be debilitating. Chronic fatigue syndrome is also referred to as myalgic encephalomyelitis (ME) or the combined term myalgic encephalomyelitis/chronic fatigue syndrome (ME/CFS), which is a complex, variable symptom, fatiguing, long-term medical condition. ME/CFS can cause a worsening of symptoms after physical or mental activity referred to as post-exertional malaise (PEM). Patients with ME/CFS also often have sleep disturbances, joint and muscle pain, cognitive impairment, and significant orthostasis. Patients suffering from ME/CFS often have a greatly lowered functional ability to complete routine activities of daily living.

Post-traumatic stress disorder (PTSD) is classified as an anxiety disorder and can also be debilitating. PTSD can develop after a person is exposed to a traumatic event, such as warfare, sexual assault, or other significant traumatic events. PTSD symptoms can include hyperarousal, irritability, anger, depression, disturbing thoughts, feelings, dreams, or other intrusive recollections of the traumatic events, and also mental or physical distress to trauma-related cues. The symptoms of PTSD can be long lasting and result in significant functional impairment.

Tourette's syndrome (TS) is a neurodevelopmental disorder characterized by multiple bodily movements, i.e. motor, tics and at least one vocal tic, i.e. phonic tics. TS typically has onset in childhood or adolescence. The tics are typically preceded by an unwanted, uncontrollable urge or sensation in the affected muscles. Examples of these tics include blinking, coughing, throat clearing, sniffing, and facial movements. Although the exact cause is unknown, it is believed that TS involves a combination of genetic and environmental factors. More specifically there may be involvement of dysfunction in the neural circuits between the basal ganglia and related structures in the brain. At present there is no cure for TS. Haloperidol (Haldol), pimozide (Orap), and aripiprazole (Abilify) are currently the only medications approved by the U.S. Food and Drug Administration (FDA) to treat tics; however, these medications all have significant long term side effects.

Parkinson's disease (PD) is a degenerative disorder of the nervous system that affects the motor system. The exact cause of the disease is unknown and may involve both genetic and environmental factors. The motor symptoms of PD include tremor, rigidity, slowness of movement, and difficulty with walking. These motor symptoms are also known as parkinsonism or parkinsonian syndrome. Also, cognitive, mood, and behavioral symptoms can be present including depression, anxiety, apathy, dementia, sleep disturbances, and sensory disturbances. The physical neurological changes associated with PD have been linked to the death of dopaminergic neurons in the substantia nigra, which is a region of the midbrain. This cell death is associated with a deficit of dopamine.

Angelman syndrome (AS), which is also known as Angelman's syndrome is a genetic disorder that affects the nervous system. Physical characteristics of the syndrome include microcephaly (i.e. a small head), In addition to physical characteristics such as a small head, telecanthus or dystopia canthorum (i.e., an increased distance between the inner corners of the eyelids), a wide mouth, and hands with tapered fingers, abnormal creases and broad thumbs The syndrome is associated with severe intellectual disability, developmental disability (e.g., a lack of functional speech), seizures (e.g. epileptic seizures), balance and movement problems, and sleep problems. Also, the electroencephalogram (EEG) of individuals with AS is usually abnormal. However, individuals with AS have a happy personality and are affectionate and seek human interaction. There is currently no cure available for AS. The seizures can be controlled by the use of one or more types of anticonvulsant medications. However, there are difficulties in ascertaining the levels and types of anticonvulsant medications needed to establish control, because people with AS often have multiple types of seizures.

Lyme disease (sometimes abbreviated LD) is an infectious disease caused by the bacteria Borrelia burgdorferi and Borrelia mayonii, carried primarily by black-legged or deer ticks. It is transmitted to the bloodstream by the bite of an infected ticks. The gram-negative bacterial species Borrelia burgdorferi, which can exist as a spirochete, is the major causative species for the disease. A common sign of a Lyme disease infection is an expanding red circular rash, known as erythema migrans, that appears at the site of the tick bite about a week after it occurred. Early symptoms of infection can include fever, headache, and tiredness. If untreated, the infection can progress to more severe neurological disorder manifestations such as loss of the ability to move one or both sides of the face, joint pain, severe headaches with neck stiffness, heart palpitations, tingling sensations, shooting pains, memory loss, and fatigue.

Coronavirus disease 2019, also known as COVID-19, is an infectious disease caused by the Severe Acute Respiratory Syndrome Corona Virus 2 (SARS-CoV-2). The disease was first identified in 2019 in Wuhan, Hubei province, China. Common symptoms of coronavirus infections include fever, cough, fatigue, shortness of breath, and loss of smell and taste. Even though the majority of cases result in mild symptoms and resolve within 2 weeks, some cases can progress to viral pneumonia, multi-organ failure, cytokine storm, and permanent tissue and organ damage, such as lung damage, heart and kidney damage, and death. The disease can be particularly serious with poor outcomes for those most at risk. Some of the more serious risk factors for severe COVID-19 illness include asthma, chronic lung disease, diabetes, serious heart conditions, chronic kidney disease being treated with dialysis, severe obesity, people aged 65 years and older, people in nursing homes or long-term care facilities, and those who are immunocompromised (such as patients undergoing cancer chemotherapy, immunologic treatments, or transplant recipients). However, there is increasing evidence of long term illness characterized by nervous system (CNS) involvement, lung/heart/renal impairment, and neurological manifestations in patients with prior COVID-19 infection. There is no direct correlation between the severity of the initial COVID-19 infection and subsequent long term sequelae. See, Ali A Asadi-Pooya and Leila Simani, Central nervous system manifestations of COVID-19: A systematic review, J Neurol Sci, 2020 Jun. 15; 413:116832. doi: 10.1016/j.jns.2020.116832. Epub 2020 Apr. 11. Many of these symptoms are associated with what is commonly known as “long COVID”, which is a condition characterized by long-term sequelae appearing or persisting after the typical convalescence period.

Antipurinergic agents constitute a family of compounds that antagonize purinergic receptors. These receptors are among the most abundant receptors in living organisms. They appeared early in evolution and are involved in regulating cellular functions. There are three known distinct classes of purinergic receptors, known as P1, P2X, and P2Y receptors. Also, purinergic signaling is a form of extracellular signaling. This signaling is mediated by purine nucleotides and nucleosides such as adenosine and adenosine triphosphate (ATP). This signaling involves the activation of purinergic receptors in the cell and/or in nearby cells, thereby regulating cellular functions. Purinergic receptors in the central nervous system play a crucial role in synaptic processes and mediating intercellular communications between neuron and glia cells, as a response to the release of adenosine triphosphate (ATP) or adenosine.

Chemical compounds that affect purinergic receptors are known. One of these is the compound, suramin, which was first synthesized in the early 1900s, and which has been found to have antipurinergic activity. Suramin is a medication used to treat the parasitic disease trypanosomiasis, which is caused by protozoa of the species Trypanosoma brucei and which is more commonly known as African sleeping sickness. The drug is also used to treat onchocerciasis, which is commonly known as river blindness. Because suramin has poor oral bioavailability, it is administered by injection into a vein. However, at the doses required for the treatment of African sleeping sickness (trypanosomiasis), suramin causes several side effects. These side effects include nausea, vomiting, diarrhea, abdominal pain, and a feeling of general discomfort. Other side effects include skin sensations such as crawling or tingling sensations, tenderness of the palms and soles, numbness of the extremities, watery eyes, rash, and photophobia. In addition, nephrotoxicity is common, as is peripheral neuropathy when the drug is administered at high doses. Regarding its pharmacokinetics, suramin is approximately 99-98% protein bound in the serum and has a half-life of 41-78 days, with an average of 50 days. Also, suramin is not extensively metabolized and is eliminated by the kidneys. Suramin is a large, polyanionic naphthylurea compound with six negative charges at physiological pH. Due to these factors, suramin cannot easily diffuse across biological membranes, which precludes it from crossing the blood-brain barrier or the blood-cerebrospinal fluid barrier. It is estimated that less than 1% of suramin crosses into the central nervous system. Therefore, for suramin to be more effectively used as a treatment for nervous system or central nervous system disorders, it would be desirable to minimize the systemic levels of suramin with a targeted delivery to brain tissue.

More recently, it has been reported that suramin exhibits an effect on several multisystem abnormalities in a mouse model of autism spectrum disorder. Also, a small human study was conducted in young boys diagnosed with autism spectrum disorder. See, Antipurinergic Therapy Corrects the Autism-Like Features in the Poly(IC) Mouse Model Robert K. Naviaux, PLoS One. 2013; 8(3): e57380, Published online 2013 Mar. 13. doi: 10.1371/journal.pone.0057380, PMCID: PMC3596371, PMID: 23516405. Also, see, PCT Patent Application Publication No. WO 2018/148580 A1, to Vaughn et al., published Aug. 16, 2018. See, also, Naviaux, R. K. et al., “Low-dose suramin in autism spectrum disorder: a small, phase I/II, randomized clinical trial”, Annals of Clinical and Translational Neurology, 2017 May 26:4(7):491-505 and R. K. Naviaux, “Antipurinergic therapy for autism—An in-depth review”, Mitochondrion 43, pp. 1-15 (2018), available online Dec. 16, 2017. For example, suramin had only ever been studied in humans for neurodevelopmental conditions as a single dose of 20 mg/kg.

From the foregoing it is apparent that the treatment of nervous system disorders remains challenging. Despite promising results from some early animal and human studies, it is recognized that much research is still needed to provide safe and effective means of the administration of antipurinergic agents, such as suramin.

Based on the limited data in the scientific literature, whether for African sleeping sickness or a nervous system disorder such as autism, there is little guidance how to select and dose these agents to achieve optimal therapeutic efficacy while minimizing undesired side effects. As will be seen from the data presented in the examples herein, the antipurinergic agent, suramin, was dosed at dosages and frequencies not previously disclosed or contemplated, and where it was discovered herein there is a dynamic, nonlinear correlation between efficacy and blood levels of the agent over time.

In the present invention, antipurinergic agents can potentially be safely and effectively administered to achieve improvements in several behavioral deficits associated with CNS disorders such as ASD, FXS, FXTAS, ME/CFS, PTSD, TS, PD, AS, and the CNS disorder manifestations associated with Lyme disease, COVID-19, other viruses (e.g. Epstein Barr Human Herpesvirus 6 and 7, Herpes Simplex Virus, Cytomegalovirus, and others), including their long term effects. The compositions and methods of administering the antipurinergic agents can provide improvements in behavioral measures of anxiety or anxiety-like behavior, willingness to explore the environment, social interaction, spatial learning and memory, irritability, agitation and/or crying, lethargy and/or social withdrawal, stereotypic behavior, hyperactivity and/or noncompliance, and restrictive and/or repetitive behaviors. Furthermore, the antipurinergic agents, can potentially be safely and effectively administered according to a pharmacokinetic and/or pharmacodynamic regimen to achieve appropriate levels of the drug. The present invention would therefore have utility for treating CNS disorders such as neurodevelopmental conditions including, but not limited to, autism spectrum disorder, FXS, FXTAS, chronic fatigue syndrome (CFS), post-traumatic stress syndrome (PTSD), Tourette's syndrome (TS), Parkinson's disease (PD), Angelman syndrome (AS), and the CNS disorder manifestations associated with Lyme disease, COVID-19, other viruses (e.g. Epstein Barr Human Herpesvirus 6 and 7, Herpes Simplex Virus, Cytomegalovirus, and others), including their long term effects.

### SUMMARY OF THE INVENTION

Compositions and methods for the treatment of nervous system disorders in mammals such as cognitive, social, or behavioral disabilities are described. These disorders include neurodevelopmental disorders such as autism spectrum disorder, FXS, FXTAS, ME/CFS, PTSD, TS, Parkinson's disease, Angelman syndrome (AS), and the CNS disorder manifestations associated with Lyme disease, COVID-19, other viruses (e.g. Epstein Barr Human Herpesvirus 6 and 7, Herpes Simplex Virus, Cytomegalovirus, and others), including their long term effects.

These compositions and methods comprise administering an effective amount of an antipurinergic agent according to a pharmacokinetic and/or pharmacodynamic method comprising an optional loading dosing regimen and a subsequent maintenance dosing regimen. Each loading dose of the loading dosing regimen may contain from about 3 mg/kg to about 30 mg/kg of the antipurinergic agent and are administered as a single dose or as multiple doses each administered with a frequency ranging from about once daily to about once every third month. Each maintenance dose of the maintenance dosing regimen may contain from about 1 mg/kg to about 15 mg/kg of the antipurinergic agent and are administered with a frequency ranging from about three times daily to about once every third month. These compositions and dosing regimens are particularly useful for maximizing therapeutic efficacy while minimizing potentially undesirable systemic side effects.

In some embodiments the present invention provides a method of treating a nervous system disorder in a mammal in need thereof, comprising administering to said mammal a pharmaceutical composition comprising an effective amount of an antipurinergic agent, or a pharmaceutically acceptable salt, ester, solvate, or prodrug thereof, according to a dosing regimen comprising (a) an optional loading dosing regimen and (b) a subsequent maintenance dosing regimen, wherein
(a) said optional loading dosing regimen is selected from (i) a single loading dose administered once, or (ii) multiple loading doses each administered with a frequency ranging from about once daily to about once every third month, wherein each loading dose comprises from about 3 mg/kg to about 30 mg/kg of the antipurinergic agent, and
(b) said subsequent maintenance dosing regimen is selected from multiple maintenance doses each administered with a frequency ranging from about three times daily to about once every third month, wherein each maintenance dose comprises from about 1 mg/kg to about 15 mg/kg of the antipurinergic agent.

In other embodiments the present invention provides a method wherein said multiple loading doses of (a) (ii) are each administered with a frequency selected from the group consisting of once daily, four times per week, three times per week, two times per week, once per week, three times per month, two times per month, once per month, once every other month, or once every third month, wherein each loading dose comprises from about 3 mg/kg to about 30 mg/kg of the antipurinergic agent, and wherein said multiple maintenance doses of (b) are each administered with a frequency selected from the group consisting of three times daily, twice daily, once daily, four times per week, three times per week, two times per week, once per week, three times per month, two times per month, once per month, once every other month, or once every third month, wherein each loading dose comprises from about 1 mg/kg to about 15 mg/kg of the antipurinergic agent.

In other embodiments the present invention provides a method wherein the molar ratio of the antipurinergic agent in each individual loading dose to the antipurinergic agent in each maintenance dose is from about 1:1.25 to about 4:1.

In other embodiments the present invention provides a method wherein the percentage of the antipurinergic agent in each individual loading dose is about 125% to about 400% of the antipurinergic agent in each maintenance dose.

In other embodiments the present invention provides a method further comprising a regimen wherein the loading dose or doses of from 3 mg/kg to about 30 mg/kg, which is defined as an initial loading dose or doses, is stepped down to one or more lower intermediate loading doses, prior to commencement of the administration of the maintenance doses.

In other embodiments the present invention provides a method wherein the molar ratio of the antipurinergic agent in each individual loading dose to the antipurinergic agent in each maintenance dose is from about 1:1.05 to about 4:1.

In other embodiments the present invention provides a method wherein the percentage of the antipurinergic agent in each individual loading dose is about 105% to about 400% of the antipurinergic agent in each maintenance dose.

In other embodiments the present invention provides a method wherein the optional loading dosing regimen is administered until a Cmin plasma level of about 8 μg/ml to 24 μg/ml of the antipurinergic agent is attained.

In other embodiments the present invention provides a method wherein the maintenance dosing regimen is continued to maintain a Cmin plasma level of about 4 μg/ml to about 18 μg/ml of the antipurinergic agent.

In other embodiments the present invention provides a method wherein the optional loading dosing regimen is administered until a Cmax plasma level of about 100 μg/ml to about 500 μg/ml, or about 150 μg/ml to about 450 μg/ml, or about 200 μg/ml to about 350 μg/ml of the antipurinergic agent is attained.

In other embodiments the present invention provides a method wherein the maintenance dosing regimen is continued to maintain a Cmax plasma level of about 50 μg/ml to about 300 μg/ml, or about 100 μg/ml to about 200 μg/ml, or about 125 μg/ml to about 175 μg/ml of the antipurinergic agent.

In other embodiments the present invention provides a method wherein the optional loading dosing regimen is administered until an AUC for the plasma level for the antipurinergic agent of about 1500 to about 7000 μg*day/L, or about 1700 to about 6500 μg*day/L, or about 2000 to about 6000 μg*day/L is attained.

In other embodiments the present invention provides a method wherein the maintenance dosing regimen is continued until an AUC for the plasma level for the antipurinergic agent of about 700 to about 3000 μg*day/L, or about 900 about 2000 μg*day/L, or about 1200 to about 1500 μg*day/L is attained.

In other embodiments the present invention provides a method wherein the mean plasma level (concentration) of the antipurinergic agent attained in the maintenance dosing regimen is about 20% to about 80% of the mean plasma level (concentration) of the antipurinergic agent attained in the loading dosing regimen.

In other embodiments the present invention provides a method wherein the mean plasma level (concentration) of the antipurinergic agent attained in the loading dosing regimen is about 125% to about 400% of the mean plasma level (concentration) of the antipurinergic agent attained in the maintenance dosing regimen.

In other embodiments the present invention provides a method wherein the Cmin plasma level (concentration) of the antipurinergic agent attained in the maintenance dosing regimen is about 20% to about 80% of the Cmin plasma level (concentration) of the antipurinergic agent attained in the loading dosing regimen.

In other embodiments the present invention provides a method wherein the Cmin plasma level (concentration) of the antipurinergic agent attained in the loading dosing regimen is about 125% to about 400% of the Cmin plasma level (concentration) of the antipurinergic agent attained in the maintenance dosing regimen.

In other embodiments the present invention provides a method wherein the Cmax plasma level (concentration) of the antipurinergic agent attained in the maintenance dosing regimen is about 20% to about 80% of the Cmax plasma level (concentration) of the antipurinergic agent attained in the loading dosing regimen.

In other embodiments the present invention provides a method wherein the Cmax plasma level (concentration) of the antipurinergic agent attained in the loading dosing regimen is about 125% to about 400% of the Cmax plasma level (concentration) of the antipurinergic agent attained in the maintenance dosing regimen.

In other embodiments the present invention provides a method wherein the AUC of the antipurinergic agent attained in the maintenance dosing regimen is about 20% to about 80% of the AUC of the antipurinergic agent attained in the loading dosing regimen.

In other embodiments the present invention provides a method wherein the AUC of the antipurinergic agent attained in the loading dosing regimen is about 125% to about 400% of the AUC of the antipurinergic agent attained in the maintenance dosing regimen.

In other embodiments the present invention provides a method wherein at least one of the following PK parameters is achieved for the optional loading dose, selected from the group consisting of a Cmin of about 8 μg/ml to about 24 μg/ml, a Cmax of about 100 μg/ml to about 500 μg/ml, or an AUC of about 1500 to about 7000 μg*day/L.

In other embodiments the present invention provides a method wherein at least one of the following PK parameters is achieved for the maintenance dose, selected from the group consisting of a Cmin of about 4 μg/ml to about 18 μg/ml, a Cmax of about 50 μg/ml to about 300 μg/ml, or an AUC of about 700 to about 3000 μg*day/L.

In other embodiments the present invention provides a method wherein the mammal is a human.

In other embodiments the present invention provides a pharmacokinetic method.

In other embodiments the present invention provides a method wherein the pharmacokinetic method is used to adjust the loading and maintenance doses according to efficacy and/or safety/tolerability endpoints.

In other embodiments the present invention provides a method wherein said efficacy endpoint is an improvement in said mammal in at least one of the following disorders, symptoms, or behavioral manifestations of the nervous disorder selected from the group consisting of
a) anxiety or anxiety-like behavior,
b) willingness to explore the environment,
c) social interaction,
d) spatial learning and memory,
e) learning and memory,
f) irritability, agitation and or crying,
g) lethargy and/or social withdrawal,
h) stereotypic behavior,
i) hyperactivity and/or noncompliance, and
j) restrictive and/or repetitive behaviors.

In other embodiments the present invention provides a method wherein said efficacy endpoint is an improvement in said mammal in at least one of the following disorders, symptoms, or behavioral manifestations of the nervous disorder selected from the group consisting of difficulty communicating, difficulty interacting with others, and repetitive behaviors.

In other embodiments the present invention provides a pharmacodynamic method.

In other embodiments the present invention provides a method wherein the pharmacodynamic method is used to adjust the loading and maintenance doses according to efficacy and/or safety/tolerability endpoints.

In other embodiments the present invention provides a method wherein said efficacy endpoint is an improvement in said mammal in at least one of the following disorders, symptoms, or behavioral manifestations of the nervous disorder selected from the group consisting of
a) anxiety or anxiety-like behavior,
b) willingness to explore the environment,
c) social interaction,
d) spatial learning and memory,
e) learning and memory,
f) irritability, agitation and or crying,
g) lethargy and/or social withdrawal,
h) stereotypic behavior,
i) hyperactivity and/or noncompliance, and
j) restrictive and/or repetitive behaviors.

In other embodiments the present invention provides a method wherein said efficacy endpoint is an improvement in said mammal in at least one of the following disorders, symptoms, or behavioral manifestations of the nervous disorder selected from the group consisting of difficulty communicating, difficulty interacting with others, and repetitive behaviors.

In other embodiments the present invention provides a method wherein the nervous system disorder is selected from the group consisting of a nervous system disorder, a psychiatric disorder, or a neurologic disorder.

In other embodiments the present invention provides a method wherein the mammal is a human.

In other embodiments the present invention provides a method wherein said nervous system, psychiatric, or neurologic disorder is selected from the group consisting of autism spectrum disorder (ASD), fragile X syndrome (FXS), fragile X-associated tremor/ataxia syndrome (FXTAS), myalgic encephalomyelitis/chronic fatigue syndrome (ME/CFS), post-traumatic stress syndrome (PTSD), Tourette's syndrome (TS), Parkinson's disease (PD), Angelman syndrome (AS), chronic Lyme disease and other nervous system disorders associated with tick-borne illnesses, and nervous system and central nervous system (CNS) disorders associated with viral infections, including their long term effects.

In other embodiments the present invention provides a method wherein the disorder is selected from ASD, FXS, FXTAS, or ME/CFS.

In other embodiments the present invention provides a method wherein the nervous system disorder is autism spectrum disorder (ASD).

In other embodiments the present invention provides a method wherein said autism spectrum disorder is selected from the group consisting of autistic disorder, childhood disintegrative disorder, pervasive developmental disorder-not otherwise specified (PDD-NOS), and Asperger syndrome.

In other embodiments the present invention provides a method wherein the disorder is FXS.

In other embodiments the present invention provides a method wherein the disorder is FXTAS.

In other embodiments the present invention provides a method wherein the disorder is ME/CFS.

In other embodiments the present invention provides a method wherein the disorder is PTSD.

In other embodiments the present invention provides a method wherein the disorder is TS.

In other embodiments the present invention provides a method wherein the disorder is PD.

In other embodiments the present invention provides a method wherein the disorder is AS.

In other embodiments the present invention provides a method wherein the disorder is a manifestation associated with Lyme disease.

In other embodiments the present invention provides a method wherein the disorder is a manifestation associated with a virus selected from the group consisting of SARS-CoV-2 (COVID-19), Epstein Barr Human Herpesvirus 6 and 7, Herpes Simplex Virus, and Cytomegalovirus, or a manifestation associated with the long term effects of the virus.

In other embodiments the present invention provides a method wherein said autism spectrum disorder manifests one or more symptoms selected from difficulty communicating, difficulty interacting with others, and repetitive behaviors.

In other embodiments the present invention provides a method that is a pharmacokinetic and/or pharmacodynamic method and is used to adjust the optional loading and maintenance doses according to efficacy endpoints based on an improvement in said human, when assessed according to the Autism Behavior Checklist (ABC), Autism Diagnostic Observation Schedule (ADOS), Autism Treatment Evaluation Checklist (ATEC), Childhood Autism Rating Scale (CARS), Clinical Global Impression (CGI) Scale, Clinical Global Impression Severity (CGI-S) Scale, Clinical Global Impression Improvement (CGI-I) Scale, or Social Responsiveness Scale (SRS).

In other embodiments the present invention provides a pharmacokinetic and/or pharmacodynamic method and is used to adjust the optional loading and maintenance doses according to efficacy endpoints based on an improvement in said human, when assessed according to the Autism Behavior Checklist (ABC).

In other embodiments the present invention provides a method wherein said antipurinergic agent is selected from the group consisting of berberine, emodin, suramin, tangeretin, A-438079, A-839977, A-804598, JNJ-47965567, and KN-62, pharmaceutically acceptable salts, esters, prodrugs, and solvates thereof, and combinations thereof.

In other embodiments the present invention provides a method wherein said antipurinergic agent is suramin, or a pharmaceutically acceptable salt, ester, solvate, or prodrug thereof.

In other embodiments the present invention provides a method wherein the pharmaceutically acceptable salt is selected from an alkali metal salt, an alkaline earth metal salt, and an ammonium salt.

In other embodiments the present invention provides a method wherein said salt is a sodium salt.

In other embodiments the present invention provides a method wherein said salt is the hexa-sodium salt.

In other embodiments the present invention provides a method wherein said composition is administered nasally or intranasally (IN).

In other embodiments the present invention provides a method wherein said composition is administered intravenously (IV).

In other embodiments the present invention provides a kit for treating a nervous system, psychiatric, or neurologic disorder in a mammal in need thereof, comprising a pharmaceutical composition comprising an effective amount of an antipurinergic agent, or a pharmaceutically acceptable salt, ester, solvate, or prodrug thereof, comprising
(a) a first component for administering the composition according to an optional loading dosing regimen, and
(b) a second component for administering the composition according to a subsequent maintenance dosing regimen.

In other embodiments the present invention provides a kit further comprising labeling instructions for administering the composition.

In other embodiments the present invention provides a kit wherein
(a) the first component for said optional loading dosing regimen is selected from
(i) a single loading dose administered once, or (ii) multiple loading doses each administered with a frequency ranging from about once daily to about once every third month, wherein each loading dose comprises from about 3 mg/kg to about 30 mg/kg of the antipurinergic agent, and
(b) the second component for said subsequent maintenance dosing regimen is selected from multiple maintenance doses each administered with a frequency ranging from about three times daily to about once every third month, wherein each maintenance dose comprises from about 1 mg/kg to about 15 mg/kg of the antipurinergic agent.

In other embodiments the present invention provides a kit wherein said multiple loading doses of (a)(ii) are each administered with a frequency selected from the group consisting of once daily, four times per week, three times per week, two times per week, once per week, three times per month, two times per month, once per month, once every other month, or once every third month, wherein each loading dose comprises from about 3 mg/kg to about 30 mg/kg of the antipurinergic agent, and said multiple maintenance doses of (b) are each administered with a frequency selected from the group consisting of three times daily, twice daily, once daily, four times per week, three times per week, two times per week, once per week, three times per month, two times per month, once per month, once every other month, or once every third month, wherein each loading dose comprises from about 1 mg/kg to about 15 mg/kg of the antipurinergic agent.

In other embodiments the present invention provides a method of inhibiting or modulating a purinergic receptor in a mammal in need thereof, comprising administering to said mammal a pharmaceutical composition comprising an effective amount of an antipurinergic agent, or a pharmaceutically acceptable salt, ester, solvate, or prodrug thereof, according to a dosing regimen comprising (a) an optional loading dosing regimen and (b) a subsequent maintenance dosing regimen, wherein
(a) said optional loading dosing regimen is selected from (i) a single loading dose administered once, or (ii) multiple loading doses each administered with a frequency ranging from about once daily to about once every third month, wherein each loading dose comprises from about 3 mg/kg to about 30 mg/kg of the antipurinergic agent, and
(b) said subsequent maintenance dosing regimen is selected from multiple maintenance doses each administered with a frequency ranging from about three times daily to about once every third month, wherein each maintenance dose comprises from about 1 mg/kg to about 15 mg/kg of the antipurinergic agent.

In other embodiments the present invention provides a method wherein said antipurinergic agent is a selective inhibitor, antagonist, or modulator of said purinergic receptor.

In other embodiments the present invention provides a method wherein said purinergic receptor is selected from the group consisting of a P1 receptor, a P2X receptor, and a P2Y receptor.

In other embodiments the present invention provides a method wherein said purinergic receptor is a P1 receptor.

In other embodiments the present invention provides a method wherein said P1 receptor is selected from a P1 receptor subtype selected from the group consisting of A₁, A_{2A}, A_{2B}, and A₃.

In other embodiments the present invention provides a method wherein said purinergic receptor is a P2X receptor.

In other embodiments the present invention provides a method wherein said P2X receptor is selected from a P2X receptor subtype selected from the group consisting of P2X₁, P2X₂, P2X₃, P2X₄, P2X₅, P2X₆, and P2X₇.

In other embodiments the present invention provides a method said P2X receptor is selected from a P2X receptor subtype selected from the group consisting of P2X₃ and P2X₇.

In other embodiments the present invention provides a method wherein said P2X receptor subtype is P2X₃.

In other embodiments the present invention provides a method wherein said P2X receptor subtype is P2X₇.

In other embodiments the present invention provides a method wherein said purinergic receptor is a P2Y receptor.

In other embodiments the present invention provides a method wherein said P2Y receptor is selected from a P2Y receptor subtype selected from the group consisting of P2Y₁, P2Y₂, P2Y₄, P2Y₆, P2Y₁₁, P2Y₁₂, P2Y₁₃, and P2Y₁₄.

In other embodiments the present invention provides a method wherein the antipurinergic agent has a selectivity of at least about two-fold (two times), or at least about five-fold (five times), or at least about ten-fold (ten times), or at least about 100-fold (ten times), or at least about 1000-fold (1000 times), or at least about 10,000-fold (10,000 times) for a P2X receptor over a P1 receptor or over a P2Y receptor.

In other embodiments the present invention provides a method wherein the antipurinergic agent has a selectivity of at least about two-fold (two times), or at least about five-fold (five times), or at least about ten-fold (ten times), or at least about 100-fold (ten times), or at least about 1000-fold (1000 times), or at least about 10,000-fold (10,000 times) for a P2Y receptor over a P1 receptor or over a P2X receptor.

In other embodiments the present invention provides a method wherein the antipurinergic agent has a selectivity of at least about two-fold (two times), or at least about five-fold (five times), or at least about ten-fold (ten times), or at least about 100-fold (ten times), or at least about 1000-fold (1000 times), or at least about 10,000-fold (10,000 times) for a P2X or a P2Y receptor over a P1 receptor.

In other embodiments the present invention provides a method wherein the antipurinergic agent has a selectivity of at least about two-fold (two times), or at least about five-fold (five times), or at least about ten-fold (ten times), or at least about 100-fold (ten times), or at least about 1000-fold (1000 times), or at least about 10,000-fold (10,000 times) for a P2X₃ receptor subtype over a P1 receptor or over a PY receptor.

In other embodiments the present invention provides a method wherein the antipurinergic agent has a selectivity of at least about two-fold (two times), or at least about five-fold (five times), or at least about ten-fold (ten times), or at least about 100-fold (ten times), or at least about 1000-fold (1000 times), or at least about 10,000-fold (10,000 times) for a P2X₇ receptor subtype over a P1 receptor or over a PY receptor.

In other embodiments the present invention provides compositions useful for practicing the recited methods herein.

In other embodiments the present invention provides the use of an antipurinergic agent or a pharmaceutically acceptable salt, ester, solvate, or prodrug thereof in the manufacture of a medicament for practicing the recited methods herein.

In other embodiments the present invention provides a kit for treating a nervous system, psychiatric, or neurologic disorder in a mammal in need thereof, comprising a pharmaceutical composition comprising an effective amount of an antipurinergic agent, or a pharmaceutically acceptable salt, ester, solvate, or prodrug thereof, comprising
(a) a first component for administering the composition according to an optional loading dosing regimen, and
(b) a second component for administering the composition according to a subsequent maintenance dosing regimen,
wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a kit further comprising labeling instructions for administering the composition comprising the antipurinergic agent, wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a kit wherein
(a) the first component for said optional loading dosing regimen is selected from
(i) a single loading dose administered once, or (ii) multiple loading doses each administered with a frequency ranging from about once daily to about once every third month, wherein each loading dose comprises from about 3 mg/kg to about 30 mg/kg of the antipurinergic agent, and
(b) the second component for said subsequent maintenance dosing regimen is selected from multiple maintenance doses each administered with a frequency ranging from about three times daily to about once every third month, wherein each maintenance dose comprises from about 1 mg/kg to about 15 mg/kg of the antipurinergic agent,
wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a kit wherein said multiple loading doses of (a)(ii) are each administered with a frequency selected from the group consisting of once daily, four times per week, three times per week, two times per week, once per week, three times per month, two times per month, once per month, once every other month, or once every third month, wherein each loading dose comprises from about 3 mg/kg to about 30 mg/kg of the antipurinergic agent, and said multiple maintenance doses of (b) are each administered with a frequency selected from the group consisting of three times daily, twice daily, once daily, four times per week, three times per week, two times per week, once per week, three times per month, two times per month, once per month, once every other month, or once every third month, wherein each loading dose comprises from about 1 mg/kg to about 15 mg/kg of the antipurinergic agent, wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a kit for treating a nervous system, psychiatric, or neurologic disorder in a mammal in need thereof, comprising a pharmaceutical composition comprising an effective amount of an antipurinergic agent, or a pharmaceutically acceptable salt, ester, solvate, or prodrug thereof, comprising
(a) a first component for administering the composition according to an optional loading dosing regimen, and
(b) a second component for administering the composition according to a subsequent maintenance dosing regimen,
wherein the antipurinergic agent is suramin.

In other embodiments the present invention provides a kit further comprising labeling instructions for administering the composition comprising the antipurinergic agent, wherein the antipurinergic agent is suramin.

In other embodiments the present invention provides a kit wherein
(a) the first component for said optional loading dosing regimen is selected from
(i) a single loading dose administered once, or (ii) multiple loading doses each administered with a frequency ranging from about once daily to about once every third month, wherein each loading dose comprises from about 3 mg/kg to about 30 mg/kg of the antipurinergic agent, and
(b) the second component for said subsequent maintenance dosing regimen is selected from multiple maintenance doses each administered with a frequency ranging from about three times daily to about once every third month, wherein each maintenance dose comprises from about 1 mg/kg to about 15 mg/kg of the antipurinergic agent,
wherein the antipurinergic agent is suramin.

In other embodiments the present invention provides a kit wherein said multiple loading doses of (a)(ii) are each administered with a frequency selected from the group consisting of once daily, four times per week, three times per week, two times per week, once per week, three times per month, two times per month, once per month, once every other month, or once every third month, wherein each loading dose comprises from about 3 mg/kg to about 30 mg/kg of the antipurinergic agent, and said multiple maintenance doses of (b) are each administered with a frequency selected from the group consisting of three times daily, twice daily, once daily, four times per week, three times per week, two times per week, once per week, three times per month, two times per month, once per month, once every other month, or once every third month, wherein each loading dose comprises from about 1 mg/kg to about 15 mg/kg of the antipurinergic agent,
wherein the antipurinergic agent is suramin.

In other embodiments the present invention provides a method of inhibiting or modulating a purinergic receptor in a mammal in need thereof, comprising administering to said mammal a pharmaceutical composition comprising an effective amount of an antipurinergic agent, or a pharmaceutically acceptable salt, ester, solvate, or prodrug thereof, according to a dosing regimen comprising (a) an optional loading dosing regimen and (b) a subsequent maintenance dosing regimen, wherein
(a) said optional loading dosing regimen is selected from (i) a single loading dose administered once, or (ii) multiple loading doses each administered with a frequency ranging from about once daily to about once every third month, wherein each loading dose comprises from about 3 mg/kg to about 30 mg/kg of the antipurinergic agent, and
(b) said subsequent maintenance dosing regimen is selected from multiple maintenance doses each administered with a frequency ranging from about three times daily to about once every third month, wherein each maintenance dose comprises from about 1 mg/kg to about 15 mg/kg of the antipurinergic agent, wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method wherein said antipurinergic agent is a selective inhibitor, antagonist, or modulator of said purinergic receptor, wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method wherein said purinergic receptor is selected from the group consisting of a P1 receptor, a P2X receptor, and a P2Y receptor, and wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method wherein said purinergic receptor is a P1 receptor, and wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method wherein said P1 receptor is selected from a P1 receptor subtype selected from the group consisting of A₁, A_{2A}, A_{2B}, and A₃, and wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method wherein said purinergic receptor is a P2X receptor, and wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method wherein said P2X receptor is selected from a P2X receptor subtype selected from the group consisting of P2X₁, P2X₂, P2X₃, P2X₄, P2X₅, P2X₆, and P2X₇, and wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method said P2X receptor is selected from a P2X receptor subtype selected from the group consisting of P2X₃ and P2X₇, and wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method wherein said P2X receptor subtype is P2X₃, and wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method wherein said P2X receptor subtype is P2X₇, and wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method wherein said purinergic receptor is a P2Y receptor, and wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method wherein said P2Y receptor is selected from a P2Y receptor subtype selected from the group consisting of P2Y₁, P2Y₂, P2Y₄, P2Y₆, P2Y₁₁, P2Y₁₂, P2Y₁₃, and P2Y₁₄, and wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method wherein the antipurinergic agent has a selectivity of at least about two-fold (two times), or at least about five-fold (five times), or at least about ten-fold (ten times), or at least about 100-fold (ten times), or at least about 1000-fold (1000 times), or at least about 10,000-fold (10,000 times) for a P2X receptor over a P1 receptor or over a P2Y receptor, wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method wherein the antipurinergic agent has a selectivity of at least about two-fold (two times), or at least about five-fold (five times), or at least about ten-fold (ten times), or at least about 100-fold (ten times), or at least about 1000-fold (1000 times), or at least about 10,000-fold (10,000 times) for a P2Y receptor over a P1 receptor or over a P2X receptor, wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method wherein the antipurinergic agent has a selectivity of at least about two-fold (two times), or at least about five-fold (five times), or at least about ten-fold (ten times), or at least about 100-fold (ten times), or at least about 1000-fold (1000 times), or at least about 10,000-fold (10,000 times) for a P2X or a P2Y receptor over a P1 receptor, wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method wherein the antipurinergic agent has a selectivity of at least about two-fold (two times), or at least about five-fold (five times), or at least about ten-fold (ten times), or at least about 100-fold (ten times), or at least about 1000-fold (1000 times), or at least about 10,000-fold (10,000 times) for a P2X₃ receptor subtype over a P1 receptor or over a PY receptor, wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method wherein the antipurinergic agent has a selectivity of at least about two-fold (two times), or at least about five-fold (five times), or at least about ten-fold (ten times), or at least about 100-fold (ten times), or at least about 1000-fold (1000 times), or at least about 10,000-fold (10,000 times) for a P2X₇ receptor subtype over a P1 receptor or over a PY receptor, wherein the antipurinergic agent is selected from the group consisting of berberine, emodin, suramin, tangeretin, A-438079, A-839977, A-804598, JNJ-47965567, and KN-62 and combinations thereof.

In other embodiments the present invention provides a method of inhibiting or modulating a purinergic receptor in a mammal in need thereof, comprising administering to said mammal a pharmaceutical composition comprising an effective amount of an antipurinergic agent, or a pharmaceutically acceptable salt, ester, solvate, or prodrug thereof, according to a dosing regimen comprising (a) an optional loading dosing regimen and (b) a subsequent maintenance dosing regimen, wherein
(a) said optional loading dosing regimen is selected from (i) a single loading dose administered once, or (ii) multiple loading doses each administered with a frequency ranging from about once daily to about once every third month, wherein each loading dose comprises from about 3 mg/kg to about 30 mg/kg of the antipurinergic agent, and
(b) said subsequent maintenance dosing regimen is selected from multiple maintenance doses each administered with a frequency ranging from about three times daily to about once every third month, wherein each maintenance dose comprises from about 1 mg/kg to about 15 mg/kg of the antipurinergic agent, wherein the antipurinergic agent is suramin.

In other embodiments the present invention provides a method wherein said antipurinergic agent is a selective inhibitor, antagonist, or modulator of said purinergic receptor, wherein the antipurinergic agent is suramin.

In other embodiments the present invention provides a method wherein said purinergic receptor is selected from the group consisting of a P1 receptor, a P2X receptor, and a P2Y receptor, and wherein the antipurinergic agent is suramin.

In other embodiments the present invention provides a method wherein said purinergic receptor is a P1 receptor, and wherein the antipurinergic agent is suramin.

In other embodiments the present invention provides a method wherein said P1 receptor is selected from a P1 receptor subtype selected from the group consisting of A₁, A_{2A}, A_{2B}, and A₃, and wherein the antipurinergic agent is suramin.

In other embodiments the present invention provides a method wherein said purinergic receptor is a P2X receptor, and wherein the antipurinergic agent is suramin.

In other embodiments the present invention provides a method wherein said P2X receptor is selected from a P2X receptor subtype selected from the group consisting of P2X₁, P2X₂, P2X₃, P2X₄, P2X₅, P2X₆, and P2X₇, and wherein the antipurinergic agent is suramin.

In other embodiments the present invention provides a method said P2X receptor is selected from a P2X receptor subtype selected from the group consisting of P2X₃ and P2X₇, and wherein the antipurinergic agent is suramin.

In other embodiments the present invention provides a method wherein said P2X receptor subtype is P2X₃, and wherein the antipurinergic agent is suramin.

In other embodiments the present invention provides a method wherein said P2X receptor subtype is P2X₇, and wherein the antipurinergic agent is suramin.

In other embodiments the present invention provides a method wherein said purinergic receptor is a P2Y receptor, and wherein the antipurinergic agent is suramin.

In other embodiments the present invention provides a method wherein said P2Y receptor is selected from a P2Y receptor subtype selected from the group consisting of P2Y₁, P2Y₂, P2Y₄, P2Y₆, P2Y₁₁, P2Y₁₂, P2Y₁₃, and P2Y₁₄, and wherein the antipurinergic agent is suramin.

In other embodiments the present invention provides a method wherein the antipurinergic agent has a selectivity of at least about two-fold (two times), or at least about five-fold (five times), or at least about ten-fold (ten times), or at least about 100-fold (ten times), or at least about 1000-fold (1000 times), or at least about 10,000-fold (10,000 times) for a P2X receptor over a P1 receptor or over a P2Y receptor, wherein the antipurinergic agent is suramin.

In other embodiments the present invention provides a method wherein the antipurinergic agent has a selectivity of at least about two-fold (two times), or at least about five-fold (five times), or at least about ten-fold (ten times), or at least about 100-fold (ten times), or at least about 1000-fold (1000 times), or at least about 10,000-fold (10,000 times) for a P2Y receptor over a P1 receptor or over a P2X receptor, wherein the antipurinergic agent is suramin.

In other embodiments the present invention provides a method wherein the antipurinergic agent has a selectivity of at least about two-fold (two times), or at least about five-fold (five times), or at least about ten-fold (ten times), or at least about 100-fold (ten times), or at least about 1000-fold (1000 times), or at least about 10,000-fold (10,000 times) for a P2X or a P2Y receptor over a P1 receptor, wherein the antipurinergic agent is suramin.

In other embodiments the present invention provides a method wherein the antipurinergic agent has a selectivity of at least about two-fold (two times), or at least about five-fold (five times), or at least about ten-fold (ten times), or at least about 100-fold (ten times), or at least about 1000-fold (1000 times), or at least about 10,000-fold (10,000 times) for a P2X₃ receptor subtype over a P1 receptor or over a PY receptor, wherein the antipurinergic agent is suramin.

In other embodiments the present invention provides a method wherein the antipurinergic agent has a selectivity of at least about two-fold (two times), or at least about five-fold (five times), or at least about ten-fold (ten times), or at least about 100-fold (ten times), or at least about 1000-fold (1000 times), or at least about 10,000-fold (10,000 times) for a P2X₇ receptor subtype over a P1 receptor or over a PY receptor, wherein the antipurinergic agent is suramin.

In other embodiments the present invention provides a pharmaceutical composition comprising an effective amount of an antipurinergic agent, or a pharmaceutically acceptable salt, ester, solvate, or prodrug thereof for use in a method for treating a nervous system disorder in a mammal in need thereof, wherein the composition is administered according to a dosing regimen comprising (a) an optional loading dosing regimen and (b) a subsequent maintenance dosing regimen, wherein
(a) said optional loading dosing regimen is selected from (i) a single loading dose administered once, or (ii) multiple loading doses each administered with a frequency ranging from about once daily to about once every third month, wherein each loading dose comprises from about 3 mg/kg to about 30 mg/kg of the antipurinergic agent, and
(b) said subsequent maintenance dosing regimen is selected from multiple maintenance doses each administered with a frequency ranging from about three times daily to about once every third month, wherein each maintenance dose comprises from about 1 mg/kg to about 15 mg/kg of the antipurinergic agent.

These and other embodiments of the present invention will become apparent from the disclosure herein.

### BRIEF DESCRIPTION OF THE DRAWINGS

FIG. 1 shows pre-dose and post-dose plasma concentrations of suramin in individual children after a 10 mg/kg dose of suramin on days 1, 28, 56, and 96 (visits 2, 4, 5, and 7) following an intravenous route of administration.

FIG. 2 shows pre-dose and post-dose plasma concentrations of suramin in individual children after a 20 mg/kg dose of suramin on days 1, 28, 56, and 96 (visits 2, 4, 5, and 7) following an intravenous route of administration.

FIG. 3 shows the pharmacokinetic parameter Cmin (μg/mL) of suramin in individual children after a 10 or 20 mg/kg dose of suramin on days 1, 28, 56, and 96 (visits 4, 5, and 7) following an intravenous route of administration versus changes in baseline Aberrant Behavior Checklist (ABC) scores. The solid line is a locally weighted (Loess) regression line representing the trend Cₘᵢₙ versus ABC scores.

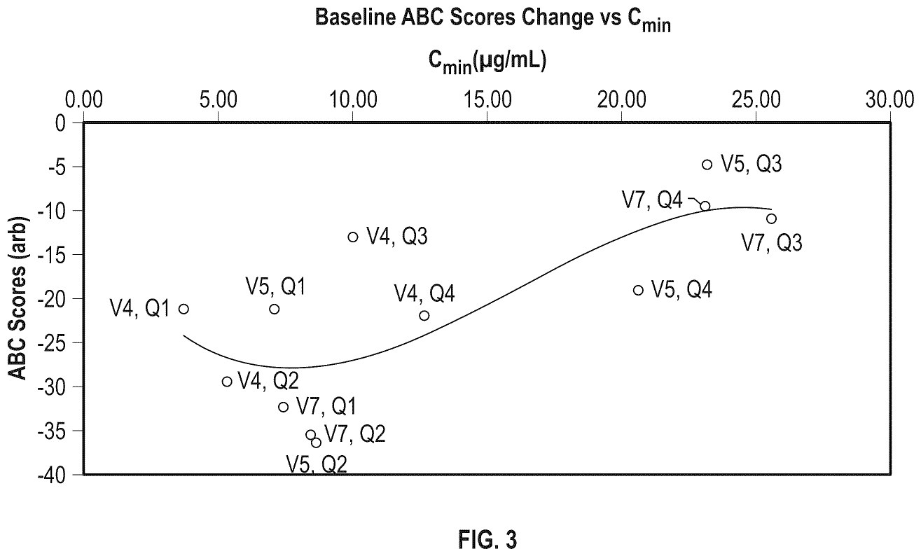

FIG. 4 shows the pharmacokinetic parameter AUC (μg*Days/mL) of suramin in individual children after a 10 or 20 mg/kg dose of suramin on days 1, 28, 56, and 96 (visits 4, 5, and 7) following an intravenous route of administration versus changes in baseline Aberrant Behavior Checklist (ABC) scores. The solid line is a locally weighted (Loess) regression line representing the trend in AUC versus ABC scores.

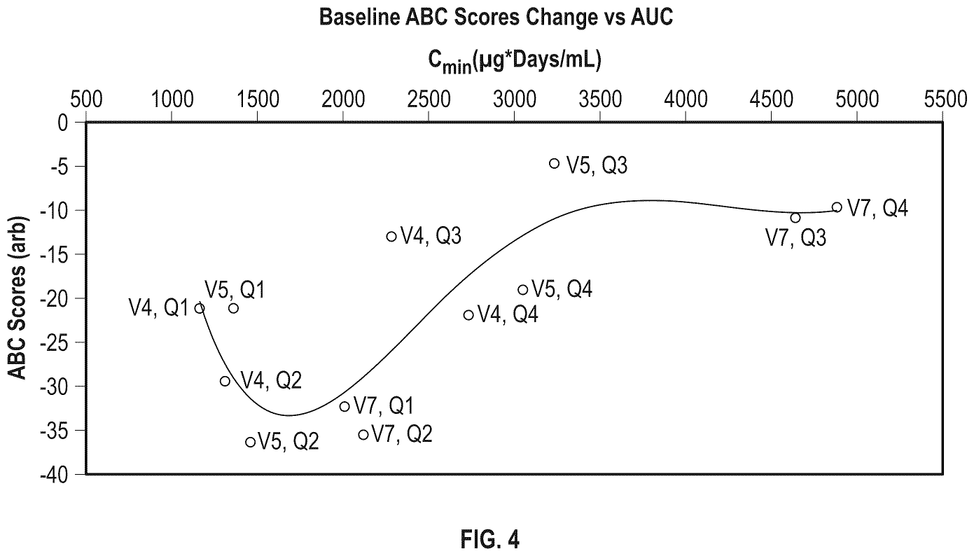

FIG. 5 shows the pharmacokinetic parameter mean quartiles Cmin (μg/mL) of suramin in individual children after a 10 or 20 mg/kg dose of suramin on days 1, 28, 56, and 96 (visits 4, 5, and 7) following an intravenous route of administration versus visits.

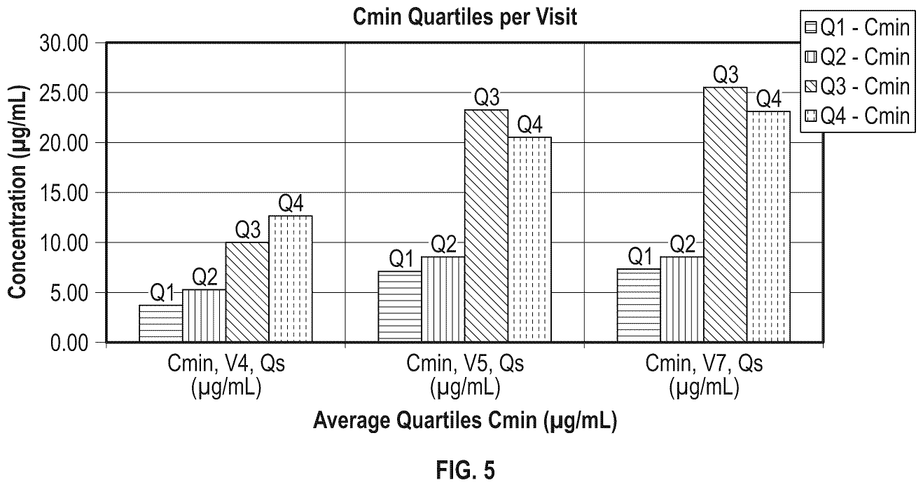

FIG. 6 shows the pharmacokinetic parameter mean quartiles AUC (μg*Days/mL) of suramin in individual children after a 10 or 20 mg/kg dose of suramin on days 1, 28, 56, and 96 (visits 4, 5, and 7) following an intravenous route of administration versus visits.

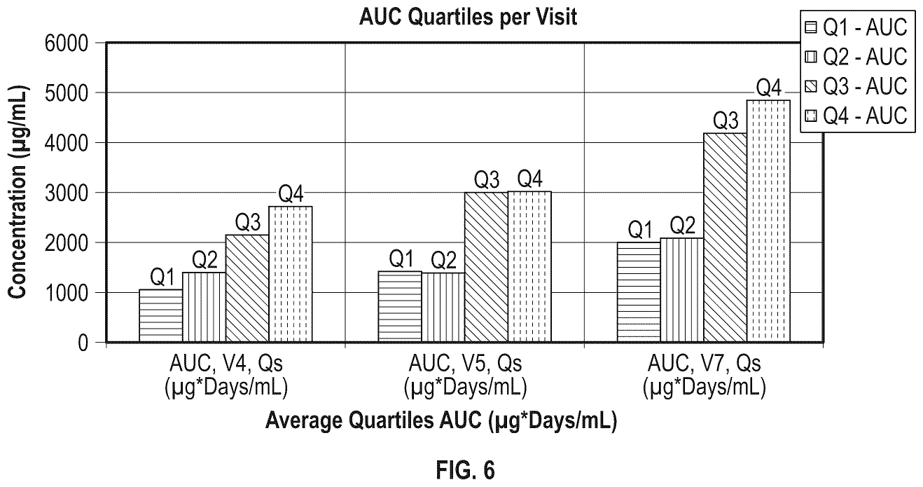

FIG. 7 shows the pharmacokinetic parameters Tmax (Days), Cmax (μg/mL), and AUC (μg*Days/mL) of suramin in individual children after a 10 mg/kg dose of suramin on days 1, 28, 56, and 96 (visits 4, 5, and 7) following an intravenous route of administration versus age (years).

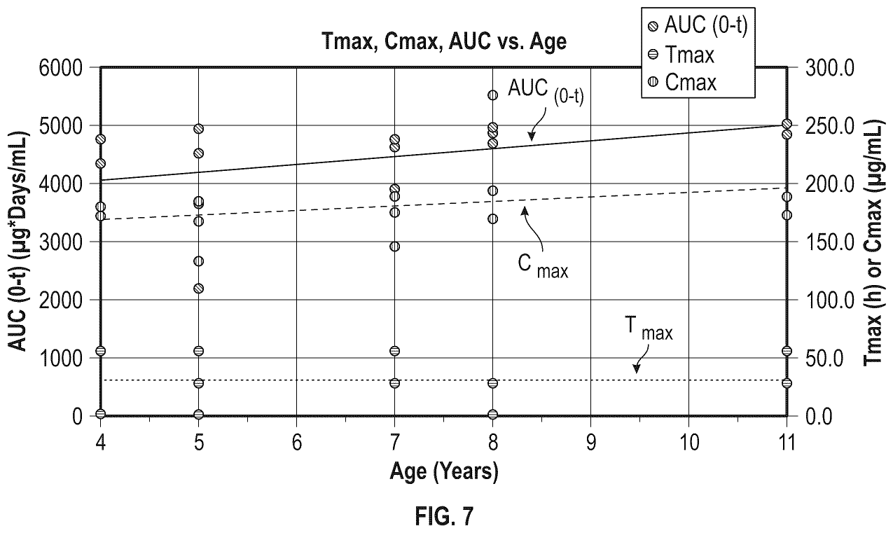

FIG. 8 shows the pharmacokinetic parameters Tmax (Days), Cmax (μg/mL), and AUC (μg*Days/mL) of suramin in individual children after a 10 mg/kg dose of suramin on days 1, 28, 56, and 96 (visits 4, 5, and 7) following an intravenous route of administration versus body max index (BMI).

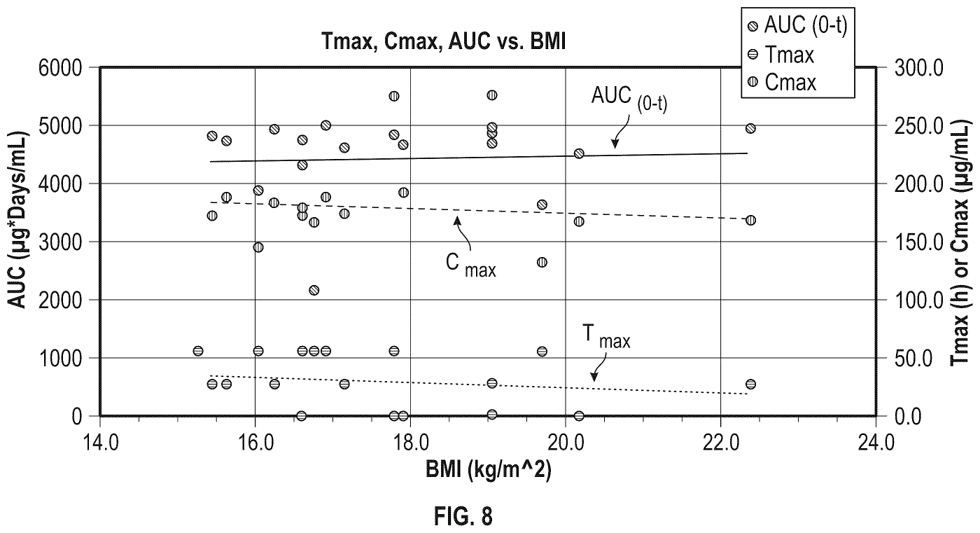

FIG. 9 shows the pharmacokinetic parameters Tmax (Days), Cmax (μg/mL), and AUC (μg*Days/mL) of suramin in individual children after a 20 mg/kg dose of suramin on days 1, 28, 56, and 96 (visits 4, 5, and 7) following an intravenous route of administration versus age (years).

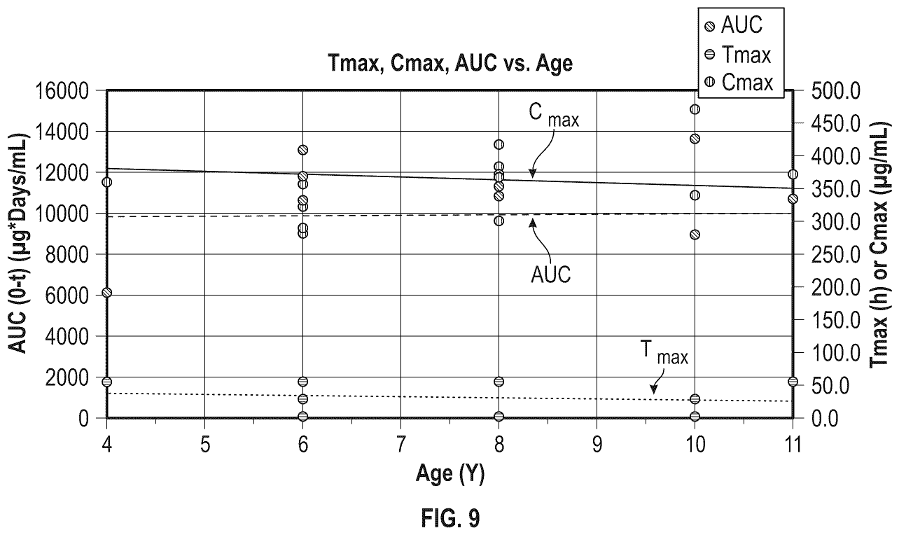

FIG. 10 shows the pharmacokinetic parameters Tmax (Days), Cmax (μg/mL), and AUC (μg*Days/mL) of suramin in individual children after a 20 mg/kg dose of suramin on days 1, 28, 56, and 96 (visits 4, 5, and 7) following an intravenous route of administration versus body mass index (BMI).

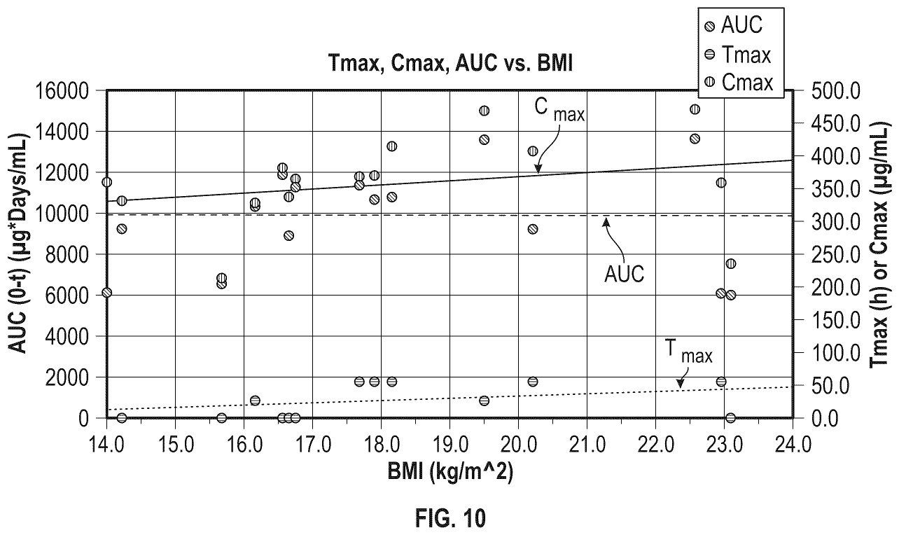

FIG. 11 shows the Aberrant Behavior Checklist core change from baseline by blood concentration level quartiles of suramin.

FIG. 12 shows the Aberrant Behavior Checklist total change from baseline by blood concentration level quartiles of suramin.

FIG. 13 shows the Aberrant Behavior Checklist core change from baseline in patients with target blood levels of suramin (8-20 μg/mL at week 14).

FIG. 14 shows the Aberrant Behavior Checklist total change from baseline in patients with target blood concentration levels of suramin (8-20 μg/mL at week 14).

FIG. 15 shows the Aberrant Behavior Checklist total change from baseline (mean±SE) by suramin blood concentration levels of suramin (BL).

### DETAILED DESCRIPTION OF THE INVENTION

### Definitions

As used herein, the following terms and abbreviations have the indicated meanings unless expressly stated to the contrary.

The term “ABC”, as used herein is also known as the “Aberrant Behavior Checklist” and is a rating scale for evaluating autism.

The term “ADOS”, as used herein is also known as “The Autism Diagnostic Observation Schedule” is an instrument for diagnosing and assessing autism. The protocol consists of a series of structured and semi-structured tasks that involve social interaction between the examiner and the person under assessment.

The term “ATEC”, as used herein is also known as “The Autism Treatment Evaluation Checklist”, is a 77-item diagnostic assessment tool that was developed at the Autism Research Institute. The ATEC was originally designed to evaluate the effectiveness of autism treatments but is also used as a screening tool.

The term “AUC”, also known as “Area Under the Curve” as used herein is standard terminology in pharmacology, specifically pharmacokinetics. The term refers to the definite integral of a curve that describes the variation of a drug concentration in blood plasma as a function of time. In practice, the drug concentration is measured at certain discrete points in time and the trapezoidal rule is used to estimate AUC. The AUC gives a measure of bioavailability and refers to the fraction of drug absorbed systemically. Knowing this, one can also determine the clearance for the drug. The AUC reflects the actual body exposure to drug after administration of a dose of the drug and is usually expressed in mg*h/L or μg*h/L (where “h” stands for hours). Alternatively, the AUC can be expressed in mg*day/L or μg*day/L. Note that the asterisk, “*”, in the units for AUC denotes a multiplication and that in alternative notations a dot “.” or multiplication symbol “x” is used. The AUC can be determined or indicated over a specified time range, such as zero to time “t” or extrapolated out to infinity, which are designated as AUC₀₋ₜ and AUC_{0-inf}, respectively.

The term “based on the suramin active” as used herein is meant to provide a basis for determining or calculating the amount of suramin based on the suramin molecular weight (i.e. a molar mass) of 1297.26 grams/mole. This is an important consideration for determining the amount of suramin when it is delivered as a salt or other form, having a different total molecular weight, such as for example the hexa-sodium salt which would have a molecular weight (i.e. a molar mass) of 1429.15 grams/mole.

The term “CARS”, as used herein is also known as “The Childhood Autism Rating Scale” and is a behavior rating scale intended to help diagnose and evaluate autism.

The term “CFS”, as used herein is also known as “Chronic Fatigue Syndrome”.

The term “CGI”, as used herein is also known as “The Clinical Global Impression” rating scale and is a measure of symptom severity, treatment response and the efficacy of treatments in treatment studies of patients with psychological disorders. Furthermore, aspects of the CGI rating scale are described as the “CGI-I” scale which stands for clinical global impression improvement scale and the “CGI-S” scale which stands for clinical global impression severity scale.

The term “Cmax” as used herein is standard terminology in pharmacology, specifically pharmacokinetics, for defining the maximum (or peak) serum concentration that a drug achieves in a specified compartment or test area of the body after the drug has been administered and before the administration of a second dose. In comparison, Cmin is the lowest concentration of a drug in the blood after a given dose.

The term “FXS” as used herein means fragile X syndrome.

The term “FXTAS” as used herein means fragile X-associated tremor/ataxia syndrome.

The term “IN” as used herein means intranasal.

The term “Loading Dosing Regimen” as used herein and described in further detail below means the part of the dosing regimen of the present invention for delivery a loading dose of the antipurinergic agent. The Loading Dosing regimen can be optional. A loading dose is described in the literature about pharmacokinetics as an initial higher dose of a drug that may be given at the beginning of a course of treatment before dropping down to a lower maintenance dose.

The term “long COVID” as used herein is also known as post-COVID-19 syndrome, post-acute sequelae of COVID-19, chronic COVID syndrome and long-haul COVID. As described in reference sources, it is a condition characterized by long-term sequelae appearing or persisting after the typical convalescence period of coronavirus disease 2019. Long COVID can affect nearly every organ system with sequelae including respiratory system disorders, nervous system and neurocognitive disorders, mental health disorders, metabolic disorders, cardiovascular disorders, gastrointestinal disorders, malaise, fatigue, musculoskeletal pain, and anemia. A wide range of symptoms are commonly discussed, including fatigue, headaches, shortness of breath, anosmia, parosmia, muscle weakness, low fever and cognitive dysfunction.

The term “long term” as used herein means a duration or manifestation of a symptom or even such as the appearance or persistence of a neurologic disorder associated with a disease or condition. For example, the manifestation or the symptom can persist for about 4 weeks to about 6 months, or in some instances even longer or chronically. Also, the appearance of the symptom might not occur or manifest for some period of time, and long term is also intended to indicate that this period can be from about 4 weeks to about 6 months or longer from the start of the underlying disease.

The term “Maintenance Dosing Regimen” as used herein and described in further detail below means the part of the dosing regimen of the present invention for delivery a maintenance dose of the antipurinergic agent. A maintenance dose is the amount of the therapeutic agent administered to maintain a desired level of the agent in the blood.

The term “ME”, as used herein is also known as “myalgic encephalomyelitis”.

The term “ME/CFS”, as used herein is also known as “myalgic encephalomyelitis/chronic fatigue syndrome”.

The term “nervous system disorder” as used herein includes the following nonlimiting, exemplary disease states, conditions or disorders selected from the group consisting of autism spectrum disorder (ASD), fragile X syndrome (FXS), fragile X-associated tremor/ataxia syndrome (FXTAS), myalgic encephalomyelitis/chronic fatigue syndrome (ME/CFS), post-traumatic stress syndrome (PTSD), Tourette's syndrome (TS), Parkinson's Disease, Angelman Syndrome (AS), and the nervous system and central nervous system (CNS) disorder manifestations often associated with Lyme disease and other tick-borne diseases, and CNS disorders associated with COVID-19, including its long term effects. The term “nervous system disorder” includes conditions that implicate the nervous system, for example conditions causing neuroinflammation.

The term “pharmaceutically acceptable” is used herein with respect to the compositions, in other words the formulations, of the present invention, and also with respect to the pharmaceutically acceptable salts, esters, solvates, and prodrugs of suramin. The pharmaceutical compositions of the present invention comprise a therapeutically effective amount of suramin and a pharmaceutically acceptable carrier. These carriers can contain a wide range of excipients. Pharmaceutically acceptable carriers are those conventionally known carriers having acceptable safety profiles. The compositions are made using common formulation techniques. See, for example, Remington's Pharmaceutical Sciences, 17^{th} edition, edited by Alfonso R. Gennaro, Mack Publishing Company, Easton, PA, 17th edition, 1985. Regarding pharmaceutically acceptable salts, these may be known and understood by a person of skill in the art and are further described below.

The term “pharmacodynamic” (PD) as used herein is used in its ordinary sense to mean the pharmacodynamic aspects of drug delivery. By definition pharmacodynamics (PD) is the study of how a drug affects an organism, e.g. the effect it has on biochemical processes of the organism.

The term “pharmacokinetic” (PK) as used herein is used in its ordinary sense to mean the pharmacokinetic aspects of drug delivery. By definition pharmacokinetics (PK) is the study of how an organism affect a drug, e.g., how and how fast it metabolizes the drug. Pharmacokinetic parameters include Cmax, Cmin, Tmax, Tmin, and AUC. Various range and ratios or combinations of these parameters can be used and designed to tailor or optimize drug delivery.

The term “PTSD”, as used herein is also known as “Post-Traumatic Stress Disorder or Syndrome”.

The term “SRS”, as used herein is also known as the “Social Responsiveness Scale” which is used herein is a measure of autism spectrum disorder.

The term “subject” means a human patient or animal in need of treatment or intervention for a nervous system disorder.

The term “therapeutically effective” means an amount of suramin needed to provide a meaningful or demonstrable benefit, as understood by medical practitioners, to a subject, such as a human patient in need of treatment. Conditions, intended to be treated include, for example, autistic disorder, childhood disintegrative disorder, pervasive developmental disorder-not otherwise specified (PDD-NOS), and Asperger syndrome. For example, a meaningful or demonstrable benefit can be assessed or quantified using various clinical parameters. The demonstration of a benefit can also include those provided by models, including but not limited to in vitro models, in vivo models, and animal models. An example of such an in vitro model is the permeation of the drug active studied using cultured human airway tissues (EpiAirway AIR-100) to simulate permeation across the nasal mucosal membrane.

The term “Tmax”, as used herein is standard terminology in pharmacology, specifically pharmacokinetics, for defining the maximum (or peak) serum concentration that a drug achieves in a specified compartment or test area of the body after the drug has been administered and before the administration of a second dose. In comparison, “Tmin” is the time at which the minimum concentration is observed.

The term “Tmin”, as used herein is standard terminology in pharmacology, specifically pharmacokinetics, for defining the minimum (trough) serum concentration that a drug achieves in a specified compartment or test area of the body after the drug has been administered and before the administration of a second dose. In comparison, “Tmax” is the time at which the maximum concentration is observed.

The term “TS”, as used herein is also known as “Tourette's syndrome”.

Routes of Administration: The U.S. Food & Drug Administration has provided a standard for a wide range of routes of administration for drugs, i.e. “Route of Administration”. For example, routes of administration include: intravenous (IV) oral, transdermal, parenteral, buccal, intracerebral, intradermal, intraepidermal, intramuscular, intraperitoneal, intrathecal, IV, percutaneous, rectal, respiratory (inhalation), and sublingual, amongst additional “other” routes, as may be appropriate.

Among other routes of administration, the standard routes of administration described by the FDA are contemplated herein as shown in the following Table 1 below (FDA Routes of Administration; retrieved from www.fda.gov; content current as of Nov. 14, 2017).

TABLE 1
FDA Routes of Administration

| | | | | NCI |
|---|---|---|---|---|
| | | SHORT | FDA | CONCEPT |
| NAME | DEFINITION | NAME | CODE | ID |
| AURICULAR (OTIC) | Administration to or by way | OTIC | 013 | C38192 |
| | of the ear. | | | |
| BUCCAL | Administration directed | BUCCAL | 030 | C38193 |
| | toward the cheek, generally | | | |
| | from within the mouth. | | | |
| CONJUNCTIVAL | Administration to the | CONJUNC | 068 | C38194 |
| | conjunctiva, the delicate | | | |
| | membrane that lines the | | | |
| | eyelids and covers the | | | |
| | exposed surface of the | | | |
| | eyeball. | | | |
| CUTANEOUS | Administration to the skin. | CUTAN | 130 | C38675 |
| DENTAL | Administration to a tooth or | DENTAL | 038 | C38197 |
| | teeth. | | | |
| ELECTRO-OSMOSIS | Administration of through | EL-OSMOS | 357 | C38633 |
| | the diffusion of substance | | | |
| | through a membrane in an | | | |
| | electric field. | | | |
| ENDOCERVICAL | Administration within the | E-CERVIC | 131 | C38205 |
| | canal of the cervix | | | |
| | uteri. Synonymous with the | | | |
| | term intracervical.. | | | |
| ENDOSINUSIAL | Administration within the | E-SINUS | 133 | C38206 |
| | nasal sinuses of the head. | | | |
| ENDOTRACHEAL | Administration directly into | E-TRACHE | 401 | C38208 |
| | the trachea. | | | |
| ENTERAL | Administration directly into | ENTER | 313 | C38209 |
| | the intestines. | | | |
| EPIDURAL | Administration upon or over | EPIDUR | 009 | C38210 |
| | the dura mater. | | | |
| EXTRA-AMNIOTIC | Administration to the outside | X-AMNI | 402 | C38211 |
| | of the membrane enveloping | | | |
| | the fetus | | | |
| EXTRACORPOREAL | Administration outside of | X-CORPOR | 057 | C38212 |
| | the body. | | | |
| HEMODIALYSIS | Administration through | HEMO | 140 | C38200 |
| | hemodialysate fluid. | | | |
| INFILTRATION | Administration that results | INFIL | 361 | C38215 |
| | substances passing into | | | |
| | tissue spaces or into cells. | | | |
| INTERSTITIAL | Administration to or in the | INTERSTIT | 088 | C38219 |
| | interstices of a tissue. | | | |
| INTRA-ABDOMINAL | Administration within the | I-ABDOM | 056 | C38220 |
| | abdomen. | | | |
| INTRA-AMNIOTIC | Administration within the | I-AMNI | 060 | C38221 |
| | amnion. | | | |
| INTRA-ARTERIAL | Administration within an | I-ARTER | 037 | C38222 |
| | artery or arteries. | | | |
| INTRA-ARTICULAR | Administration within a | I-ARTIC | 007 | C38223 |
| | joint. | | | |
| INTRABILIARY | Administration within the | I-BILI | 362 | C38224 |
| | bile, bile ducts or | | | |
| | gallbladder. | | | |
| INTRABRONCHIAL | Administration within a | I-BRONCHI | 067 | C38225 |
| | bronchus. | | | |
| INTRABURSAL | Administration within a | I-BURSAL | 025 | C38226 |
| | bursa. | | | |
| INTRACARDIAC | Administration with the | I-CARDI | 027 | C38227 |
| | heart. | | | |
| INTRACARTILAGINOUS | Administration within a | I-CARTIL | 363 | C38228 |
| | cartilage; endochondral. | | | |
| INTRACAUDAL | Administration within the | I-CAUDAL | 413 | C38229 |
| | cauda equina. | | | |
| INTRACAVERNOUS | Administration within a | I-CAVERN | 132 | C38230 |
| | pathologic cavity, such as | | | |
| | occurs in the lung in | | | |
| | tuberculosis. | | | |
| INTRACAVITARY | Administration within a non- | I-CAVIT | 023 | C38231 |
| | pathologic cavity, such as | | | |
| | that of the cervix, uterus, or | | | |
| | penis, or such as that which | | | |
| | is formed as the result of a | | | |
| | wound. | | | |
| INTRACEREBRAL | Administration within the | I-CERE | 404 | C38232 |
| | cerebrum. | | | |
| INTRACISTERNAL | Administration within the | I-CISTERN | 405 | C38233 |
| | cisterna magna | | | |
| | cerebellomedularis. | | | |
| INTRACORNEAL | Administration within the | I-CORNE | 406 | C38234 |
| | cornea (the transparent | | | |
| | structure forming the | | | |
| | anterior part of the fibrous | | | |
| | tunic of the eye). | | | |
| INTRACORONAL, | Administration of a drug | I-CORONAL | 117 | C38217 |
| DENTAL | within a portion of a tooth | | | |
| | which is covered by enamel | | | |
| | and which is separated from | | | |
| | the roots by a slightly | | | |
| | constricted region known as | | | |
| | the neck. | | | |
| INTRACORONARY | Administration within the | I-CORONARY | 119 | C38218 |
| | coronary arteries. | | | |
| INTRACORPORUS | Administration within the | I-CORPOR | 403 | C38235 |
| CAVERNOSUM | dilatable spaces of the | | | |
| | corporus cavernosa of the | | | |
| | penis. | | | |
| INTRADERMAL | Administration within the | I-DERMAL | 008 | C38238 |
| | dermis. | | | |
| INTRADISCAL | Administration within a disc. | I-DISCAL | 121 | C38239 |
| INTRADUCTAL | Administration within the | I-DUCTAL | 123 | C38240 |
| | duct of a gland. | | | |
| INTRADUODENAL | Administration within the | I-DUOD | 047 | C38241 |
| | duodenum. | | | |
| INTRADURAL | Administration within or | I-DURAL | 052 | C38242 |
| | beneath the dura. | | | |
| INTRAEPIDERMAL | Administration within the | I-EPIDERM | 127 | C38243 |
| | epidermis. | | | |
| INTRAESOPHAGEAL | Administration within the | I-ESO | 072 | C38245 |
| | esophagus. | | | |
| INTRAGASTRIC | Administration within the | I-GASTRIC | 046 | C38246 |
| | stomach. | | | |
| INTRAGINGIVAL | Administration within the | I-GINGIV | 307 | C38247 |
| | gingivae. | | | |
| INTRAILEAL | Administration within the | I-ILE | 365 | C38249 |
| | distal portion of the small | | | |
| | intestine, from the jejunum | | | |
| | to the cecum. | | | |
| INTRALESIONAL | Administration within or | I-LESION | 042 | C38250 |
| | introduced directly into a | | | |
| | localized lesion. | | | |
| INTRALUMINAL | Administration within the | I-LUMIN | 310 | C38251 |
| | lumen of a tube. | | | |
| INTRALYMPHATIC | Administration within the | I-LYMPHAT | 352 | C38252 |
| | lymph. | | | |
| INTRAMEDULLARY | Administration within the | I-MEDUL | 408 | C38253 |
| | marrow cavity of a bone. | | | |
| INTRAMENINGEAL | Administration within the | I-MENIN | 409 | C38254 |
| | meninges (the three | | | |
| | membranes that envelope the | | | |
| | brain and spinal cord). | | | |
| INTRAMUSCULAR | Administration within a | IM | 005 | C28161 |
| | muscle. | | | |
| INTRAOCULAR | Administration within the | I-OCUL | 036 | C38255 |
| | eye. | | | |
| INTRAOVARIAN | Administration within the | I-OVAR | 354 | C38256 |
| | ovary. | | | |
| INTRAPERICARDIAL | Administration within the | I-PERICARD | 314 | C38257 |
| | pericardium. | | | |
| INTRAPERITONEAL | Administration within the | I-PERITON | 004 | C38258 |
| | peritoneal cavity. | | | |
| INTRAPLEURAL | Administration within the | I-PLEURAL | 043 | C38259 |
| | pleura. | | | |
| INTRAPROSTATIC | Administration within the | I-PROSTAT | 061 | C38260 |
| | prostate gland. | | | |
| INTRAPULMONARY | Administration within the | I-PULMON | 414 | C38261 |
| | lungs or its bronchi. | | | |
| INTRASINAL | Administration within the | I-SINAL | 010 | C38262 |
| | nasal or periorbital sinuses. | | | |
| INTRASPINAL | Administration within the | I-SPINAL | 022 | C38263 |
| | vertebral column. | | | |
| INTRASYNOVIAL | Administration within the | I-SYNOV | 019 | C38264 |
| | synovial cavity of a joint. | | | |
| INTRATENDINOUS | Administration within a | I-TENDIN | 049 | C38265 |
| | tendon. | | | |
| INTRATESTICULAR | Administration within the | I-TESTIC | 110 | C38266 |
| | testicle. | | | |
| INTRATHECAL | Administration within the | IT | 103 | C38267 |
| | cerebrospinal fluid at any | | | |
| | level of the cerebrospinal | | | |
| | axis, including injection into | | | |
| | the cerebral ventricles. | | | |
| INTRATHORACIC | Administration within the | I-THORAC | 006 | C38207 |
| | thorax (internal to the ribs); | | | |
| | synonymous with the term | | | |
| | endothoracic. | | | |
| INTRATUBULAR | Administration within the | I-TUBUL | 353 | C38268 |
| | tubules of an organ. | | | |
| INTRATUMOR | Administration within a | I-TUMOR | 020 | C38269 |
| | tumor. | | | |
| INTRATYMPANIC | Administration within the | I-TYMPAN | 366 | C38270 |
| | aurus media. | | | |
| INTRAUTERINE | Administration within the | I-UTER | 028 | C38272 |
| | uterus. | | | |
| INTRAVASCULAR | Administration within a | I-VASC | 021 | C38273 |
| | vessel or vessels. | | | |
| INTRAVENOUS | Administration within or | IV | 002 | C38276 |
| | into a vein or veins. | | | |
| INTRAVENOUS BOLUS | Administration within or | IV BOLUS | 138 | C38274 |
| | into a vein or veins all at | | | |
| | once. | | | |
| INTRA VENOUS DRIP | Administration within or | IV DRIP | 137 | C38279 |
| | into a vein or veins over a | | | |
| | sustained period of time. | | | |
| INTRA VENTRICULAR | Administration within a | I-VENTRIC | 048 | C38277 |
| | ventricle. | | | |
| INTRA VESICAL | Administration within the | I-VESIC | 128 | C38278 |
| | bladder. | | | |
| INTRA VITREAL | Administration within the | I-VITRE | 311 | C38280 |
| | vitreous body of the eye. | | | |
| IONTOPHORESIS | Administration by means of | ION | 055 | C38203 |
| | an electric current where | | | |
| | ions of soluble salts migrate | | | |
| | into the tissues of the body. | | | |
| IRRIGATION | Administration to bathe or | IRRIG | 032 | C38281 |
| | flush open wounds or body | | | |
| | cavities. | | | |
| LARYNGEAL | Administration directly upon | LARYN | 364 | C38282 |
| | the larynx. | | | |
| NASAL | Administration to the nose; | NASAL | 014 | C38284 |
| | administered by way of the | | | |
| | nose. | | | |
| NASOGASTRIC | Administration through the | NG | 071 | C38285 |
| | nose and into the stomach, | | | |
| | usually by means of a tube. | | | |
| NOT APPLICABLE | Routes of administration are | NA | 312 | C48623 |
| | not applicable. | | | |
| OCCLUSIVE DRESSING | Administration by the | OCCLUS | 134 | C38286 |
| TECHNIQUE | topical route which is then | | | |
| | covered by a dressing which | | | |
| | occludes the area. | | | |
| OPHTHALMIC | Administration to the | OPHTHALM | 012 | C38287 |
| | external eye. | | | |
| ORAL | Administration to or by way | ORAL | 001 | C38288 |
| | of the mouth. | | | |
| OROPHARYNGEAL | Administration directly to | ORO | 410 | C38289 |
| | the mouth and pharynx. | | | |
| OTHER | Administration is different | OTHER | 135 | C38290 |
| | from others on this list. | | | |
| PARENTERAL | Administration by injection, | PAREN | 411 | C38291 |
| | infusion, or implantation. | | | |
| PERCUTANEOUS | Administration through the | PERCUT | 113 | C38676 |
| | skin. | | | |
| PERIARTICULAR | Administration around a | P-ARTIC | 045 | C38292 |
| | joint. | | | |
| PERIDURAL | Administration to the outside | P-DURAL | 050 | C38677 |
| | of the dura mater of the | | | |
| | spinal cord.. | | | |
| PERINEURAL | Administration surrounding | P-NEURAL | 412 | C38293 |
| | a nerve or nerves. | | | |
| PERIODONTAL | Administration around a | P-ODONT | 040 | C38294 |
| | tooth. | | | |
| RECTAL | Administration to the | RECTAL | 016 | C38295 |
| | rectum. | | | |
| RESPIRATORY | Administration within the | RESPIR | 136 | C38216 |
| (INHALATION) | respiratory tract by inhaling | | | |
| | orally or nasally for local or | | | |
| | systemic effect. | | | |
| RETROBULBAR | Administration behind the | RETRO | 034 | C38296 |
| | pons or behind the eyeball. | | | |
| SOFT TISSUE | Administration into any soft | SOFT TIS | 109 | C38198 |
| | tissue. | | | |
| SUBARACHNOID | Administration beneath the | S-ARACH | 066 | C38297 |
| | arachnoid. | | | |
| SUBCONJUNCTIVAL | Administration beneath the | S-CONJUNC | \|096 | C38298 |
| | conjunctiva. | | | |
| SUBCUTANEOUS | Administration beneath the | SC | 003 | C38299 |
| | skin; hypodermic. | | | |
| | Synonymous with the term | | | |
| | SUBDERMAL. | | | |
| SUBLINGUAL | Administration beneath the | SL | 024 | C38300 |
| | tongue. | | | |
| SUBMUCOSAL | Administration beneath the | S-MUCOS | 053 | C38301 |
| | mucous membrane. | | | |
| TOPICAL | Administration to a | TOPIC | 011 | C38304 |
| | particular spot on the outer | | | |
| | surface of the body. The E2B | | | |
| | term TRANSMAMMARY | | | |
| | is a subset of the term | | | |
| | TOPICAL. | | | |
| TRANSDERMAL | Administration through the | T-DERMAL | 358 | C38305 |
| | dermal layer of the skin to | | | |
| | the systemic circulation by | | | |
| | diffusion. | | | |
| TRANSMUCOSAL | Administration across the | T-MUCOS | 122 | C38283 |
| | mucosa. | | | |
| TRANSPLACENTAL | Administration through or | T-PLACENT | 415 | C38307 |
| | across the placenta. | | | |
| TRANSTRACHEAL | Administration through the | T-TRACHE | 355 | C38308 |
| | wall of the trachea. | | | |
| TRANSTYMPANIC | Administration across or | T-TYMPAN | 124 | C38309 |
| | through the tympanic cavity. | | | |
| UNASSIGNED | Route of administration has | UNAS | 400 | C38310 |
| | not yet been assigned. | | | |
| UNKNOWN | Route of administration is | UNKNOWN | 139 | C38311 |
| | unknown. | | | |
| URETERAL | Administration into the | URETER | 112 | C38312 |
| | ureter. | | | |
| URETHRAL | Administration into the | URETH | 017 | C38271 |
| | urethra. | | | |
| VAGINAL | Administration into the | VAGIN | 015 | C38313 |
| | vagina. | | | |

The term “step down” as used herein with respect to the dosing regimens means, that the dose of the antipurinergic agent is gradually decreased from the initial loading dose of the loading dosing regimen to the final maintenance dose of the maintenance dosing regimen. The decrease or “step down” can involve one or more intermediate loading doses that constitute a lower dose of the drug active than in the initial loading dose, but at a level that is higher than in the maintenance dose. This “step down” is in contrast to direct “drop down” dosing where there is an administration of the loading dose or doses followed by a direct drop to the maintenance dose, and no intermediate doses.

The terms “treat,” “treating” or “treatment,” as used herein, include alleviating, abating or ameliorating the condition, e.g. autism and other nervous system disorders, or preventing or reducing the risk of developing the condition or exhibiting the symptoms of the condition, ameliorating or preventing the underlying causes of the symptoms, inhibiting the condition, arresting the development of the condition, relieving the condition, causing regression of the condition, or stopping the symptoms of the condition, either prophylactically and/or therapeutically.

The methods of treatment using the antipurinergic agent or a pharmaceutically acceptable salt, ester, solvate, or prodrug thereof or the pharmaceutical compositions of the present invention, in various embodiments also include the use of the antipurinergic agent or a pharmaceutically acceptable salt, ester, solvate, or prodrug thereof in the manufacture of a medicament for the desired treatment, such as for a nervous system disorder.

### Purinergic Receptors

Purinergic receptors (also “purinoceptors”) are a family of membrane receptors found in most mammalian tissues. There are three known distinct classes of purinergic receptors, known as P1, P2X, and P2Y receptors (also known as P1, P₂X, and P₂Y, respectively). Each of the classes of these receptors further comprises receptor subtypes which are coded for by distinct genes. The P1 class has the following subtypes: A₁, A_{2B}, A_{2B}, and A₃. The P2X class has the following subtypes: P2X₁, P2X₂, P2X₃, P2X₄, P2X₅, P2X₆, and P2X₇. The P2Y class has the following subtypes: P2Y₁, P2Y₂, P2Y₄, P2Y₆, P2Y₁₁, P2Y₁₂, P2Y₁₃, and P2Y₁₄.

The P2X and P2Y receptors are expressed in cells of the human central nervous system. P2X receptors are ATP-gated non-selective cation channels that mediate fast excitatory transmission in diverse regions of the brain and spinal cord. The P2X₇ receptor subtype is a ligand-gated, non-selective, cation channel that is a member of the P2X superfamily (P2X₁₋₇ subtypes) of purinoreceptors. The human P2X₇ receptor was first cloned in 1997 and has since received significant attention from a number of research groups in both academia and industry for its potential role in a number of neurological and neurodegenerative disorders. The P2X₇ receptor was originally described in cells of hematopoietic origin (macrophages, microglia, and certain lymphocytes), and are also found on cells of the nervous system such as neurons, astrocytes, oligodendrocytes, and Schwann cells. Activation of the P2X₇ receptor results in flux of small cations (Na⁺, Ca²⁺, and K⁺), the release of proinflammatory cytokines IL-1b and IL-18, as well as a number of downstream events. The P2X₇ receptor is activated by high concentrations of ATP, which is released in large quantities following cell injury. While pharmacological blockade of P2X₇ receptors have been studied in animal models of neurological disorders, much is unknown about their effects and implications. See, Rachael Bartlett, Leanne Stokes and Ronald Sluyter. P2X₇ Antagonists in Models of Disease. Pharmacological Reviews Jul. 1, 2014, 66 (3) 638-675; Sperlagh, B and Illes, P. The P2X₇ purinergic receptor: from physiology to neurological disorders. Trends in Pharmacological Sciences, October 2014, Vol 35, No 10; and Skaper, S. D., Debetto, P., Giusti, P. The P2X₇ purinergic receptor: from physiology to neurological disorders. FASEB J. 24, 337-345 (2010).

In the present invention it is found that the administration of antipurinergic agents having an inhibitory, antagonizing, or modulating effect on one of more these receptors could be useful for treatment of a nervous system disorder.

### Antipurinergic Agents

Purinergic signaling is an extracellular process mediated by purine nucleotides and nucleosides such as adenosine and ATP. This process involves the activation of purinergic receptors in the cell and/or in nearby cells, thereby regulating cellular functions. The present invention is based on administering compounds with antipurinergic activity, such as antagonists to treat or ameliorate the symptoms and manifestations associated with nervous system disorders. Nonlimiting examples of antipurinergic agents useful in the present invention include those selected from the group consisting of berberine, emodin, suramin, tangeretin, A-438079, A-839977, A-804598, JNJ-47965567, KN-62, and combinations thereof. Also, pharmaceutically acceptable salts, esters, prodrugs, solvates (including hydrates), and polymorphs are contemplated within the scope of the present invention. In other embodiments, the antipurinergic agent can be administered in combination with other drugs, potentiators, adjuvants, penetration enhancers, and the like.

### Berberine

In some embodiments, the present invention utilizes a therapeutically effective amount of berberine, which is believed to have potential antipurinergic activity. Berberine is a quaternary ammonium salt alkaloid compound from the protoberberine group of benzylisoquinoline alkaloids found in plants as Berberis, for example, Berberis vulgaris (barberry). The compound is typically isolated as a quaternary ammonium salt as indicated by the structure below.

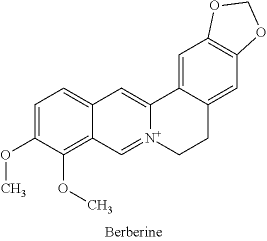 [SMILES: COc1ccc2cc3[n+](cc2c1OC)CCc1cc2c(cc1-3)OCO2]

Berberine corresponds to the CAS Registry Number 2086-83-1 and ChemSpider ID 2263. Berberine is a yellow solid corresponding to the chemical formula C₂₀H₁₈NO₄ and has a molar mass of 336.361 grams/mole. Note that these molecular weight values will vary slightly depending on what atomic weight values are used for the calculations. One of the chemical names for berberine is: Benzo[g]-1,3-benzodioxolo[5,6-a]quinolizinium, 5,6-dihydro-9,10-dimethoxy-.

### Emodin

In some embodiments, the present invention utilizes a therapeutically effective amount of the antipurinergic agent emodin, or a pharmaceutically acceptable salt, ester, solvate, or prodrug thereof for treating a nervous system disorder.

Emodin is a hydroxyanthraquinone that is an orange solid at room temperature that is found in rhubarb and buckthorn. Emodin corresponds to the CAS Registry Number 518-82-1 and ChemSpider ID 3107. The IUPAC name for emodin is: 1,3,8-Trihydroxy-6-methylanthracene-9,10-dione

The chemical formula of emodin is C₁₅H₁₀O. Emodin therefore has a molecular weight (i.e. a molar mass) of 270.240 grams/mole. Note that these molecular weight values will vary slightly depending on what atomic weight values are used for the calculations. The chemical structure for emodin is shown below.

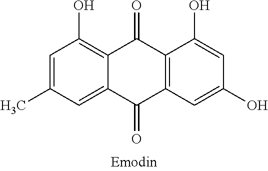 [SMILES: Cc1cc(O)c2c(c1)C(=O)c1cc(O)cc(O)c1C2=O]

Pharmaceutically acceptable salts, esters, solvates, and prodrugs of emodin are useful for the methods and compositions of the present invention. As used herein, “pharmaceutically acceptable salts, esters, solvates and prodrugs” refer to derivatives of emodin.

The pharmaceutically acceptable salts, esters, solvates and prodrugs of emodin can be prepared from the parent compound by conventional chemical methods. Generally, the salts can be prepared by reacting the compound with a stoichiometric amount of the appropriate base in water or in an organic solvent, or in a mixture of the two; generally, non-aqueous media like ether, ethyl acetate, ethanol, isopropanol, or acetonitrile are preferred. The pharmaceutically acceptable esters of emodin can be prepared by reaction with a carboxylic acid and removal of water. For example, one or more of the three phenolic groups can be esterified to form for example acetate groups.

The solvates of emodin means that one or more solvent molecules are associated with one or more molecules of emodin, including fraction solvates such as, e.g., 0.5 and 2.5 solvates. The solvents can be selected from a wide range of solvents including water, ethanol, isopropanol, and the like. The prodrugs of emodin can be prepared using convention chemical methods, depending on the prodrug chosen. A prodrug is a medication or compound that, after administration, is metabolized (i.e., converted within the body) into a pharmacologically active drug. Prodrugs can be designed to improve bioavailability when a drug itself is poorly absorbed from the gastrointestinal tract. Prodrugs are intended to include covalently bonded carriers that release an active parent drug of the present invention in vivo when such prodrug is administered. In some classifications, esters are viewed as prodrugs.

In some embodiments of the present invention, it has been found advantageous to co-administer the emodin with piperine. Piperine is an alkaloid responsible for the pungency of black pepper and long pepper. Piperine has the IUPAC name (2E,4E)-5-(2H-1,3-Benzodioxol-5-yl)-1-(piperidin-1-yl)penta-2,4-dien-1-one and corresponds to the CAS registry number 94-62-2 and ChemSpider number 553590. Piperine has the molecular formula C₁₇H₁₉NO₃ and a molar mass of 285.343 grams/mole and corresponds to the following chemical structure shown below.

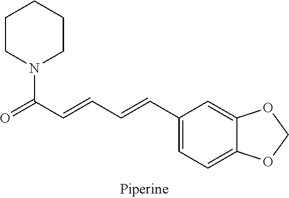 [SMILES: O=C(/C=C/C=C/c1ccc2c(c1)OCO2)N1CCCCC1]

In some embodiments the emodin and piperine are administered in a 1 to 1 weight ratio. Other weight ratios for the emodin to piperine can range from about 100 to 1 to about 1 to 100, or from about 10 to 1 to about 1 to 10, or from about 1 to 5 to about 5 to 1, or from about 1 to 2 to about 2 to 1, or from about 1 to 1.5 to about 1.5 to 1.

### Suramin

In some embodiments, the present invention utilizes a therapeutically effective amount of the antipurinergic agent suramin, or a pharmaceutically acceptable salt, ester, solvate, or prodrug thereof for treating a nervous system disorder.

Suramin is a sulfonic acid drug compound, corresponding to the CAS Registry Number 145-63-1 and ChemSpider ID 5168. One of the chemical names for suramin is: 1,3,5-Naphthalenetrisulfonic acid, 8,8′-[carbonylbis[imino-3,1-phenylenecarbonylimino(4-methyl-3,1-phenylene)carbonylimino]]bis-. The compound is a medication used to treat African sleeping sickness (trypanosomiasis) and river blindness (onchocerciasis) and is known by the trade names Antrypol, 309 F, 309 Fourneau, Bayer 205, Germanin, Moranyl, Naganin, and Naganine. However, the drug is not approved by the US FDA. The drug is administered by venous injection.

Suramin is reported to have a half-life of between about 41 to 78 days with an average of 50 days. See, Phillips, Margaret A.; Stanley, Jr, Samuel L. (2011). “Chapter 50: Chemotherapy of Protozoal Infections: Amebiasis, Giardiasis, Trichomoniasis, Trypanosomiasis, Leishmaniasis, and Other Protozoal Infections”. In Brunton, Laurence L. Chabner, Bruce A.; Knollmann, Bjorn Christian (eds.). Goodman and Gilman's The Pharmacological Basis of Therapeutics (12th ed.). McGraw Hill. pp. 1437-1438.

The chemical formula of suramin is C₅₁H₄₀N₆O₂₃S₆. Suramin therefore has a molecular weight (i.e. a molar mass) of 1297.26 grams/mole. Suramin is usually delivered as a sodium sulfonate salt, such as the hexa-sodium salt, which has a molecular weight (i.e. a molar mass) of 1429.15 grams/mole. Note that these molecular weight values will vary slightly depending on what atomic weight values are used for the calculations. The chemical structure for suramin is shown below.

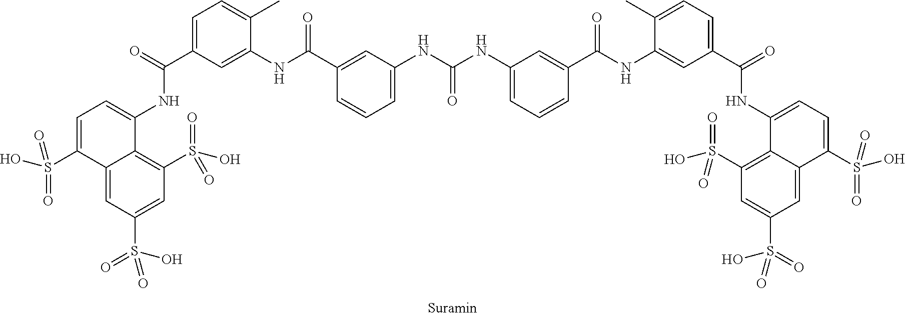 [SMILES: Cc1ccc(C(=O)Nc2ccc(S(=O)(=O)O)c3cc(S(=O)(=O)O)cc(S(=O)(=O)O)c23)cc1NC(=O)c1cccc(NC(=O)Nc2cccc(C(=O)Nc3cc(C(=O)Nc4ccc(S(=O)(=O)O)c5cc(S(=O)(=O)O)cc(S(=O)(=O)O)c45)ccc3C)c2)c1]

Pharmaceutically acceptable salts, esters, solvates, and prodrugs of suramin are useful for the methods and compositions of the present invention. As used herein, “pharmaceutically acceptable salts, esters, solvates, and prodrugs” refer to derivatives of suramin. Examples of pharmaceutically acceptable salts include, but are not limited to, alkali metal salts, alkaline earth metal salts, and ammonium salts. Examples of alkali metal salts include lithium, sodium, and potassium salts. Examples of alkaline earth metal salts include calcium and magnesium salts. The ammonium salt, NH4⁺. itself can be prepared, as well as various monoalkyl, dialkyl, trialkyl, and tetraalkyl ammonium salts. Also, one or more of the alkyl groups of such ammonium salts can be further substituted with groups such as hydroxy groups, to provide an ammonium salt of an alkanol amine. Ammonium salts derived from diamines such as 1,2-diaminoethane are contemplated herein. The hexa-sodium salt of suramin is useful herein.

The pharmaceutically acceptable salts, esters, solvates, and prodrugs of suramin can be prepared from the parent compound by conventional chemical methods. Generally, the salts can be prepared by reacting the free acid form of the compound with a stoichiometric amount of the appropriate base in water or in an organic solvent, or in a mixture of the two; generally, non-aqueous media like ether, ethyl acetate, ethanol, isopropanol, or acetonitrile are preferred. The esters of suramin can be prepared by reacting the parent compound with an alcohol, and removal of water formed from the reaction. Alternatively, other methods can be used. Anywhere from one up to all six of the sulfonate groups of suramin can be esterified to form a mono-ester up to a hexa-ester sulfonate.

The solvates of suramin means that one or more solvent molecules are associated with one or more molecules of suramin, including fraction solvates such as, e.g., 0.5 and 2.5 solvates. The solvents can be selected from a wide range of solvents including water, ethanol, isopropanol, and the like. The prodrugs of suramin can be prepared using convention chemical methods, depending on the prodrug chosen. A prodrug is a medication or compound that, after administration, is metabolized (i.e., converted within the body) into a pharmacologically active drug. Prodrugs can be designed to improve bioavailability when a drug itself is poorly absorbed from the gastrointestinal tract. Prodrugs are intended to include covalently bonded carriers that release an active parent drug of the present invention in vivo when such prodrug is administered. In some classifications, esters are viewed as prodrugs, such as the esters of suramin described herein. Other types of prodrugs can include sulfonamide derivatives and anhydrides.

Furthermore, the various esters and prodrugs can include further derivatization to make polyethylene glycol (PEG) and polypropylene glycol (PPG) derivatives and mixed derivatives, an example of which would a pegylated derivative.

### Tangeretin

In some embodiments, the present invention utilizes a therapeutically effective amount of the antipurinergic agent tangeretin, or a pharmaceutically acceptable solvate or prodrug thereof for treating a nervous system disorder.

Tangeretin is an O-polymethoxylated flavone that is found in tangerine and other citrus peels and is used as a dietary supplement. Tangeretin corresponds to the CAS Registry Number 481-53-8 and ChemSpider ID 61389. The IUPAC name for tangeretin is: 5,6,7,8-tetramethoxy-2-(4-methoxyphenyl)-4H-1-benzopyran-4-one.

The chemical formula of tangeretin is C₂₀H₂₀O₇. A-804598 therefore has a molecular weight (i.e. a molar mass) of 372.37 grams/mole. Note that these molecular weight values will vary slightly depending on what atomic weight values are used for the calculations. The chemical structure for tangeretin is shown below.

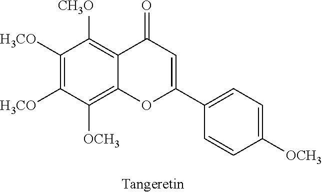

Pharmaceutically acceptable solvates, and prodrugs of tangeretin are useful for the methods and compositions of the present invention. As used herein, “pharmaceutically acceptable solvates and prodrugs” refer to derivatives of tangeretin.

The pharmaceutically acceptable solvates and prodrugs of tangeretin can be prepared from the parent compound by conventional chemical methods. The solvates of tangeretin means that one or more solvent molecules are associated with one or more molecules of tangeretin, including fraction solvates such as, e.g., 0.5 and 2.5 solvates. The solvents can be selected from a wide range of solvents including water, ethanol, isopropanol, and the like. The prodrugs of tangeretin can be prepared using convention chemical methods, depending on the prodrug chosen. A prodrug is a medication or compound that, after administration, is metabolized (i.e., converted within the body) into a pharmacologically active drug. Prodrugs can be designed to improve bioavailability when a drug itself is poorly absorbed from the gastrointestinal tract. Prodrugs are intended to include covalently bonded carriers that release an active parent drug of the present invention in vivo when such prodrug is administered. In some classifications, amides are viewed as prodrugs.

A-438079

In some embodiments, the present invention utilizes a therapeutically effective amount of the antipurinergic agent A-438079 (also known as “A438079” or “A 438079”), or a pharmaceutically acceptable salt, solvate, or prodrug thereof for treating a nervous system disorder. For example, the hydrochloride salt of A-438079 is especially useful.

A-438079 is reported to have activity as a P2X₇ receptor antagonist against the P2X₇ receptor, both human and rat. The compound corresponds to the chemical formula C₁₃H₉Cl₂N₅ and has a molar mass of 306.15 grams/mole (the monohydrochloride salt has a molar mass of 342.6 grams/mole). Note that these molecular weight values will vary slightly depending on what atomic weight values are used for the calculations. One of the chemical names for A-438079 is 3-[[5-(2,3-dichlorophenyl)-1H-tetrazol-1-yl]methyl]-pyridine, monohydrochloride. The compound corresponds to the CAS Registry Number 899431-18-6. The compound is also known to form hydrates. The structure for the hydrochloride salt is depicted below.

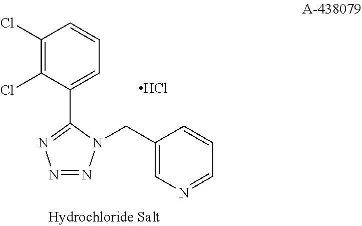 [SMILES: Clc1cccc(-c2nnnn2Cc2cccnc2)c1Cl ; Cl]

A-839977

In some embodiments, the present invention utilizes a therapeutically effective amount of the antipurinergic agent A-839977 (also known as “A839977” or “A 4839977”), or a pharmaceutically acceptable salt, solvate, or prodrug thereof for treating a nervous system disorder.

A-839977 is reported to have activity as a P2X₇ receptor antagonist. The compound corresponds to the chemical formula CB H9CLN5 and has a molar mass of 413.26 grams/mole. Note that these molecular weight values will vary slightly depending on what atomic weight values are used for the calculations. One of the chemical names for A-839977 1-(2,3-Dichlorophenyl)-N-[2-(pyridin-2-yloxy)benzyl]-1H-tetrazol-5-amine. The compound corresponds to the CAS Registry Number 870061-27-1 and the chemical structure is shown below.

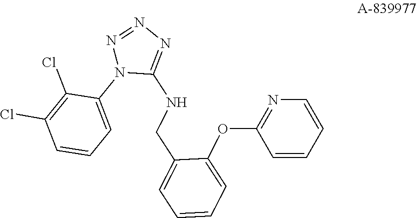 [SMILES: Clc1cccc(-n2nnnc2NCc2ccccc2Oc2ccccn2)c1Cl]

A-804598

In some embodiments, the present invention utilizes a therapeutically effective amount of the antipurinergic agent A-804598 (also known as “A804598” or “A 804598”), or a pharmaceutically acceptable salt, solvate, or prodrug thereof for treating a nervous system disorder.

A-804598 is a cyano guanidine P2X₇ inhibitor corresponding to the CAS Registry Number 1125758-85-1 and ChemSpider ID 26377919. One of the chemical names for A-804598 is N-Cyano-N″-[(1S)-1-phenylethyl]-N′-5-quinolinyl-guanidine. The compound is described as a central nervous system penetrant, competitive and selective P2X₇ receptor antagonist with IC50s of 9 nM, 10 nM and 11 nM for mouse, rat and human P2X₇ receptors, respectively.

The chemical formula of A-804598 is C₁₉H₁₇N₅. A-804598 therefore has a molecular weight (i.e. a molar mass) of 315.372 grams/mole. Note that these molecular weight values will vary slightly depending on what atomic weight values are used for the calculations. The chemical structure for A-804598 is shown below.

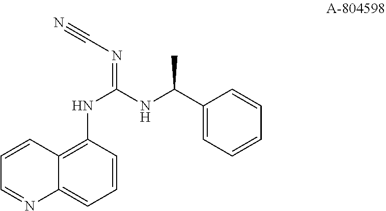 [SMILES: C[C@H](N/C(=N/C#N)Nc1cccc2ncccc12)c1ccccc1]

Pharmaceutically acceptable salts, amides, solvates, and prodrugs of A-804598 are useful for the methods and compositions of the present invention. As used herein, “pharmaceutically acceptable salts, amides, solvates, and prodrugs” refer to derivatives of A-804598. Examples of pharmaceutically acceptable salts include, but are not limited to strong acid salts such as the hydrochloride, hydrobromide, hydroiodide, sulfate, and hydrogen sulfate salts.

The pharmaceutically acceptable salts, amides, solvates, and prodrugs of A-804598 can be prepared from the parent compound by conventional chemical methods. Generally, the salts can be prepared by reacting the free base form of the compound with a stoichiometric amount of the appropriate acid in water or in an organic solvent, or in a mixture of the two; generally, non-aqueous media like ether, ethyl acetate, ethanol, isopropanol, or acetonitrile are preferred. The amides can be prepared by reacting the parent compound with a carboxylic acid.

The solvates of A-804598 means that one or more solvent molecules are associated with one or more molecules of A-804598, including fraction solvates such as, e.g., 0.5 and 2.5 solvates. The solvents can be selected from a wide range of solvents including water, ethanol, isopropanol, and the like. The prodrugs of A-804598 can be prepared using convention chemical methods, depending on the prodrug chosen. A prodrug is a medication or compound that, after administration, is metabolized (i.e., converted within the body) into a pharmacologically active drug. Prodrugs can be designed to improve bioavailability when a drug itself is poorly absorbed from the gastrointestinal tract. Prodrugs are intended to include covalently bonded carriers that release an active parent drug of the present invention in vivo when such prodrug is administered. In some classifications, amides are viewed as prodrugs.

### JNJ-47965567

In some embodiments, the present invention utilizes a therapeutically effective amount of the antipurinergic agent JNJ-47965567, or a pharmaceutically acceptable salt, solvate, or prodrug thereof for treating a nervous system disorder. The compound was shown to suppress epileptic seizures in a mouse model of epilepsy.

JNJ-47965567 is a selective P2X₇ antagonist, corresponding to the CAS Registry Number 1428327-31-4. One of the chemical names for JNJ-47965567 is 2-(Phenylthio)-N-[[tetrahydro-4-(4-phenyl-1-piperazinyl)-2H-pyran-4-yl]methyl]-3-pyridinecarboxamide. The chemical formula of JNJ-47965567 is C₂₈H₃₂N₄O₂S. The compound has a molecular weight (i.e. a molar mass) of 488.64 grams/mole. Note that these molecular weight values will vary slightly depending on what atomic weight values are used for the calculations.

The chemical structure for JNJ-47965567 is shown below.

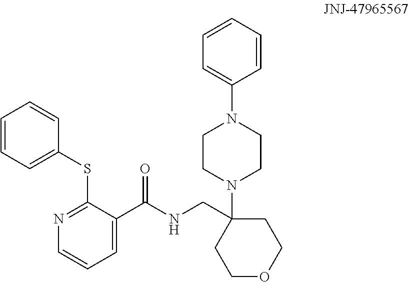 [SMILES: O=C(NCC1(N2CCN(c3ccccc3)CC2)CCOCC1)c1cccnc1Sc1ccccc1]

### KN-62

In some embodiments, the present invention utilizes a therapeutically effective amount of the antipurinergic agent KN-62, or a pharmaceutically acceptable salt, solvate, or prodrug thereof for treating a nervous system disorder. KN-62 is a derivative of isoquinolinesulfonamide and is reported to inhibit the P2X₇ receptor.

KN-62 corresponds to the CAS Registry Number 127191-97-3 and the ChemSpider ID 4471558. The IUPAC name for JNJ-47965567 is 4-[(2S)-2-[(5-isoquinolinylsulfonyl)methylamino]-3-oxo-3-(4-phenyl-1-piperazinyl)propyl] phenyl isoquinolinesulfonic acid ester.

The chemical formula of KN-62 is C₃₈H₃₅N₅O₆S₂ and has a molecular weight (i.e. a molar mass) of 721.84 grams/mole. Note that these molecular weight values will vary slightly depending on what atomic weight values are used for the calculations. The chemical structure for KN-62 is shown below.

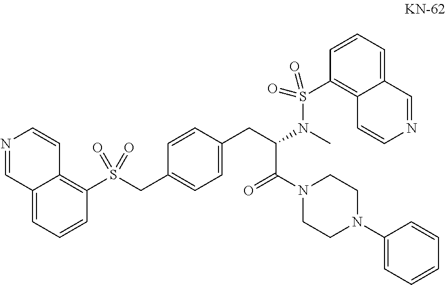 [SMILES: CN([C@@H](Cc1ccc(CS(=O)(=O)c2cccc3cnccc23)cc1)C(=O)N1CCN(c2ccccc2)CC1)S(=O)(=O)c1cccc2cnccc12]

### Dosing Regimen

Based on the limited data in the scientific literature, whether for African sleeping sickness or a nervous system disorder such as autism, there is little guidance how to select and dose these agents to achieve optimal therapeutic efficacy while minimizing undesired side effects. For example, suramin had only ever been studied in humans for neurodevelopmental conditions at a single dose of 20 mg/kg. See, R. K. Naviaux, “Antipurinergic therapy for autism—An in-depth review”, Mitochondrion 43, pp. 1-15 (2018), available online Dec. 16, 2017. Based on the data and outcomes from these studies, it was surprising to discover that optimal clinical efficacy was achieved based on a dynamic and non-linear correlation between clinical efficacy and blood levels. Given the linear correlation between clinical efficacy and blood levels that has been observed after a single administration of suramin, the methods and compositions of the present invention would not have been expected.

As seen from the data presented in the examples of the present invention, the antipurinergic agent, suramin, was dosed at dosages and frequencies not previously disclosed or contemplated leading to the discovery of a dynamic, nonlinear correlation between efficacy and blood levels of the agent over time.

The present invention provides compositions and methods for treating nervous system disorders in a mammal. These compositions and methods comprise administering an effective amount of an antipurinergic agent according to a pharmacokinetic and/or pharmacodynamic dosing regimen. This dosing regimen comprises an optional loading dosing regimen and a subsequent maintenance dosing regimen. Each loading dose of the loading dosing regimen contains from about 3 mg/kg to about 30 mg/kg of the antipurinergic agent and is administered as a single dose or as multiple doses each administered with a frequency ranging from about once daily to about once every third month. The maintenance doses of the maintenance dosing regimen each contain from about 1 mg/kg to about 15 mg/kg of the antipurinergic agent and are administered with a frequency ranging from about three times daily to about once every third month. These compositions and dosing regimens are particularly useful for maximizing therapeutic efficacy while minimizing potentially undesirable systemic side effects.

Because many of the antipurinergic agents to be delivered have relatively long half-lives and/or protein binding and because it is highly desirable to maximize efficacy while minimizing systemic side effects, the present invention utilizes a dosing regimen taking into account both pharmacokinetic and pharmacodynamic considerations. For example, suramin is approximately 99-98% protein bound in the serum and has a half-life of 41-78 days with an average of 50 days. The dosing regimen of the present invention comprises a loading dosing regimen, which can be optional, and a subsequent maintenance dosing regimen.

### Loading Dosing Regimen

The Loading Dosing regimen for delivery of the antipurinergic agent can be optional. As stated above, a loading dose is described in the literature about pharmacokinetics as an initial higher dose of a drug that may be given at the beginning of a course of treatment before dropping or stepping down to a lower maintenance dose.

The optional loading dosing regimen can be selected from (i) delivery of a single loading dose administered once, or (ii) delivery of multiple loading doses each administered with a frequency ranging from about once daily to about once every third month, wherein each loading dose comprises from about 3 mg/kg to about 30 mg/kg of the antipurinergic agent. In some embodiments, each loading dose may independently comprise from about 3 mg/kg to about 30 mg/kg of the antipurinergic agent. In some embodiments, each loading dose may comprise the same or approximately the same amount of the antipurinergic agent, which can range from about 3 mg/kg to about 30 mg/kg of the antipurinergic agent.

In further embodiments the loading dosing regimen the multiple loading doses are each administered with a frequency selected from the group consisting of once daily, four times per week, three times per week, two times per week, once per week, three times per month, two times per month, once per month, once every other month, or once every third month, wherein each loading dose comprises from about 3 mg/kg to about 30 mg/kg of the antipurinergic agent.

Whether the loading dosing regimen is utilized, i.e. is optional, is within the skill and medical judgment of the prescribing health care professional, based on the antipurinergic agent and the patient.

Other ranges for each dose of the loading dosing regimen can comprise from about 5 mg/kg to about 25 mg/kg, and from about 10 mg/kg to about 20 mg/kg. Other ranges and non-integer values of the antipurinergic agent can be selected.

These values can be based on the active chemical agent of the anti-purinergic agent, to account for differences in salt and pro-drug forms.

### Maintenance Dosing Regimen

After administration of the antipurinergic agent according to the optional loading dosing regimen, the agent is then subsequently administered according to a maintenance dosing regimen. As stated above, a maintenance dose is the amount of the therapeutic agent administered to maintain a desired level of the agent in the blood.

The maintenance dosing regimen is administered with multiple maintenance doses each administered with a frequency ranging from about three times daily to about once every third month, wherein each maintenance dose comprises from about 1 mg/kg to about 15 mg/kg of the antipurinergic agent

In further embodiments the maintenance dosing regimen is selected such that multiple maintenance doses of the antipurinergic agent are each administered with a frequency selected from the group consisting of three times daily, twice daily, once daily, four times per week, three times per week, two times per week, once per week, three times per month, two times per month, once per month, once every other month, or once every third month, wherein each loading dose comprises from about 1 mg/kg to about 15 mg/kg of the antipurinergic agent

The selection of an appropriate maintenance dosing regimen is within the skill and medical judgment of the prescribing health care professional, based on the antipurinergic agent and the patient.

Other ranges for each dose of the loading dosing regimen can comprise from about 2 mg/kg to about 12 mg/kg, and from about 5 mg/kg to about 10 mg/kg. Other ranges and non-integer values of the antipurinergic agent can be selected.

These values can be based on the active chemical agent of the anti-purinergic agent, to account for molar mass differences in salt and pro-drug forms

### Compositions

Compositions of the antipurinergic agent can also be determined on a weight basis. In one embodiment the compositions useful here comprise from about 50% to about 99.99% by weight of the antipurinergic agent or a pharmaceutically salt, ester, solvate or, prodrug thereof, based on the weight of the antipurinergic agent active. In another embodiment these compositions here comprise from about 1% to about 25% by weight of the antipurinergic agent or a pharmaceutically salt, ester, solvate or, prodrug thereof, based on the weight of the antipurinergic agent active.

For compositions comprising a designated amount or weight percentage of the antipurinergic agent, the agent is determined or calculated based on the actual amount of the antipurinergic agent moiety, based on the molar mass, and not including the additional weight provided by any counter ions, or ester, solvate or prodrug moieties when a salt, ester, solvate, or prodrug is used. In other words, the compositions are based on the amount or weight percentage of the antipurinergic agent chemical moiety.

### Methods of Treatment and Dosing Regimens

The present invention utilizes a therapeutically effective amount of the antipurinergic agent or a pharmaceutically acceptable salt thereof and a pharmaceutically acceptable carrier for administering of the antipurinergic agent for treating a nervous system disorder such as autism spectrum disorders, FXS, FXTAS, ME/CFS, PTSD, TS, PD, AS, or the CNS disorder manifestations associated with Lyme disease, COVID-19, other viruses (e.g. Epstein Barr Human Herpesvirus 6 and 7, Herpes Simplex Virus, Cytomegalovirus, and others), including their long term effects.

The methods comprise amongst others, as may be appropriate, the following routes of administration for the antipurinergic agent, or a pharmaceutically acceptable salt, ester, solvate, or prodrug thereof to a human patient, in need thereof: IV, oral, transdermal, parenteral, buccal, intracerebral, intradermal, intraepidermal, intramuscular, intraperitoneal, intrathecal, nasal, other, percutaneous, rectal, respiratory (inhalation), and sublingual.

Various dosing regimens can be prescribed in accordance with the present invention and used based on the skill and knowledge of the physician or other practitioner. Based on the pharmacokinetic and pharmacodynamic parameters of the antipurinergic agent, the dosing amount and regimen can be appropriately varied.

Therapy can be continued in the judgment of the physician or practitioner until the desired therapeutic benefit is achieved. In many instances, it is desirable to continue long term or maintenance therapy.

### Evaluation of Treatments

The present invention provides a method wherein the nervous system disorder, such as autism spectrum disorder, FXS, FXTAS, CFS, PTSD, TS, PD, AS, or a nervous system disorder manifestation associated with Lyme disease or COVID-19 and other viruses (e.g. Epstein Barr Human Herpesvirus 6 and 7, Herpes Simplex Virus, Cytomegalovirus, and others), including their long term effects, includes one or more symptoms selected from difficulty communicating, difficulty interacting with others, disruptive and repetitive behaviors, motor tics, and phonic tics. With these disorders, the patient can often exhibit one or more symptoms or behavioral manifestations, or study endpoints selected from the group consisting of
a) anxiety or anxiety-like behavior,
b) willingness to explore the environment,
c) social interaction,
d) spatial learning and memory,
e) learning and memory,
f) irritability, agitation and or crying,
g) lethargy and/or social withdrawal,
h) stereotypic behavior,
i) hyperactivity and/or noncompliance, or
j) restrictive and/or repetitive behaviors.

Patients with autism spectrum disorder, FXS, FXTAS, CFS, PTSD, TS, PD, AS, or the CNS disorder manifestations associated with Lyme disease, COVID-19, other viruses (e.g. Epstein Barr Human Herpesvirus 6 and 7, Herpes Simplex Virus, Cytomegalovirus, and others), including their long term effects can be evaluated using a variety of rating scales to determine the level of severity of their disorder and any improvements or changes upon administration of a treatment.

For example, the present invention provides a method wherein treating the autism spectrum disorder, FXS, FXTAS, CFS, PTSD, TS, PD, AS, or the CNS disorder manifestations associated with Lyme disease, COVID-19, other viruses (e.g. Epstein Barr Human Herpesvirus 6 and 7, Herpes Simplex Virus, Cytomegalovirus, and others), including their long term effects. comprises improving more or more symptoms of the patient relative to the symptoms prior to therapy. The improvement can be determined by comparing an assessment score of the patient's symptoms relative to a score from the patient's symptoms prior to said administration. It is desirable to provide an improvement of 10% or more relative to a score from the patient prior to administration of the treatment.

Examples of assessment scales for evaluating autism spectrum disorder include those selected from ABC, ADOS, ATEC, CARS CGI (CGI-S and CGI-I), and SRS.

The term “ABC” is also known as the “Aberrant Behavior Checklist” and is a rating scale for evaluating autism. On this scale, the lower the score the greater the improvement. The term “ADOS” is also known as “The Autism Diagnostic Observation Schedule”. The protocol consists of a series of structured and semi-structured tasks that involve social interaction between the examiner and the person under assessment. The term “ATEC” is also known as “The Autism Treatment Evaluation Scale” and is a 77-item diagnostic assessment tool that was developed at the Autism Research Institute. The ATEC was originally designed to evaluate the effectiveness of autism treatments, but is also used as a screening tool. The term “CARS” is also known as “The Childhood Autism Rating Scale” and is a behavior rating scale intended to help diagnose and evaluate autism. The term “CGI” is also known as “The Clinical Global Impression” rating scale and is a measure of symptom severity, treatment response and the efficacy of treatments in treatment studies of patients with psychological disorders. The term “SRS” is also known as the “Social Responsiveness Scale” which is used herein and is a measure of autism spectrum disorder.

For example, the present invention provides a method wherein an ADOS score of the patient is improved by 1.6 or more relative to a score prior to administration of treatment, or a corresponding performance improvement on a similar test. Furthermore, the present invention provides a method wherein the p-value of improvement of ADOS score or similar test is 0.05 or less. In another aspect, the present invention provides a method wherein the size effect of improvement of the ADOS score or similar test is about 1 or more or is up to about 2.9 or more.

See, Aman M G, Singh N N, Stewart A W, Field C J. The aberrant behavior checklist: a behavior rating scale for the assessment of treatment effects. Am J Ment Defic. 1985 March; 89(5):485-91. PMID: 3993694; and Kaat, A. J., Lecavalier, L. & Aman, M. G. Validity of the Aberrant Behavior Checklist in Children with Autism Spectrum Disorder. J Autism Dev Disorder 44, 1103-1116 (2014). https://doi.org/10.1007/s10803-013-1970-0].

### Formulations

In one embodiment, the compositions or formulations of the present invention comprise an antipurinergic agent or a pharmaceutically acceptable salt, ester, solvate, or prodrug thereof and a pharmaceutically acceptable carrier. These formulations can be prepared using standard formulation and mixing techniques familiar to one of ordinary skill in the art of pharmaceuticals and formulations.

The antipurinergic agent can be selected from the group consisting of berberine, emodin, suramin, tangeretin, A-438079, A-839977, A-804598, JNJ-47965567, and KN-62, pharmaceutically acceptable salts, esters, prodrugs, and solvates thereof, and combinations thereof.

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating.

Pharmaceutical compositions may comprise one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present teachings can be administered intravenously, by injection, orally, parenterally, or via other routes of administration, neat or in combination with conventional pharmaceutical carriers. Applicable carriers can include one or more substances which can also act as flavoring agents, lubricants, solubilizers, suspending agents, fillers, glidants, compression aids, binders or tablet-disintegrating agents, or encapsulating materials. Oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, ion exchange resins, benzyl alcohol, eucalyptol, gelatin, limonene, mannitol, menthol, menthone, menthyl acetate, sucralose, and vanillin. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions for oral or parenteral administration (such as intravenous, intramuscular, or other injections), including suspensions, emulsions, syrups, elixirs, and additionally for inhaled delivery. A compound of the present teachings can be dissolved or suspended in a pharmaceutically acceptable liquid carrier such as water, an organic solvent, or a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile injectable solutions can also be administered intravenously. Compositions for oral administration can be in either liquid or solid form.

Compounds described herein can be administered parenterally or intraperitoneally. Solutions or suspensions of these compounds or pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

The pharmaceutical composition can be in the form of a unit dosage, for example, as vials, ampoules, tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the pharmaceutical composition formulated, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some instances, it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

The pharmaceutical compositions herein can comprise a penetration enhancer. Surprisingly, the following penetration enhancers have been found to increase the transmucosal tissue penetration of suramin: methyl Beta-cyclodextrin, caprylocaproyl macrogol-8 glycerides, and 2-(2-ethoxyethoxy)ethanol. The material methyl Beta-cyclodextrin (methyl-beta-cyclodextrin) is also known by the CAS Registry Number 128446-36-6 and the trade name methyl betadex. The material caprylocaproyl macrogol-8 glycerides is also known as caprylocaproyl polyoxyl-8 glycerides and PEG-8 caprylic/capric glycerides, by the CAS Registry Number 85536-07-8, and the trade name Labrasol®. The material 2-(2-ethoxyethoxy)ethanol is also known as diethylene glycol ethyl ether, by the CAS Registry Number 111-90-0, and by the trade names Carbitol™ and Transcutol® P. The penetration enhance is generally used at about 40% by weight of the composition. Other useful ranges are from about 0.1% to about 90% by weight of the composition, or from about 1% to about 80% by weight of the composition, or from about 10% to about 75% by weight of the composition, or from about 25% to about 50% by weight of the composition.

The water in the composition is usually Q.S. The abbreviation QS stands for Quantum Satis and means to add as much of the ingredient, in this case water, to achieve the desired result, but not more.

Other ingredients can include various salts for osmolality control and thickening agents.

In one aspect, the pharmaceutical composition is selected from a solution, suspension, or dispersion for administration as a spray or aerosol. In other aspects the formulation can be delivered as drops by a nose dropper or applied directly to the nasal cavity. Other pharmaceutical compositions are selected from the group consisting of a gel, ointment, lotion, emulsion, cream, foam, mousse, liquid, paste, jelly, or tape, that is applied to the nasal cavity.

Useful herein are compositions wherein the pharmaceutically acceptable carrier is selected from water or mixtures of water with other water-miscible components. In the case of emulsions, the components do not have to be miscible with water.

In other embodiments the compositions can comprise a buffer to maintain the pH of the drug formulation, a pharmaceutically acceptable thickening agent, humectant and surfactant. Buffers that are suitable for use in the present invention include, for example, hydrochloride, acetate, citrate, carbonate and phosphate buffers.

The viscosity of the compositions of the present invention can be maintained at a desired level using a pharmaceutically acceptable thickening agent. Thickening agents that can be used in accordance with the present invention include for example, xanthan gum, carbomer, polyvinyl alcohol, alginates, acacia, chitosans, sodium carboxyl methylcellulose (Na CMC) and mixtures thereof. The concentration of the thickening agent will depend upon the agent selected and the viscosity desired.

In the present invention, any other suitable absorption enhancers as known in the art may also be used.

The compositions can also comprise an absorption enhancing ingredient such as (i) a surfactant; (ii) a bile salt (including sodium taurocholate); (iii) a phospholipid additive, mixed micelle, or liposome; (iv) an alcohol (including a polyol as discussed above, for example, propylene glycol or polyethylene glycol such as PEG 3000, etc.); (v) an enamine; (vi) a nitric oxide donor compound; (vii) a long-chain amphipathic molecule; (viii) a small hydrophobic uptake enhancer; (ix) sodium or a salicylic acid derivative; (x) a glycerol ester of acetoacetic acid; (xi) a cyclodextrin or cyclodextrin derivative; (xii) a medium-chain or short-chain (e.g. Cl to C 12) fatty acid; and (xiii) a chelating agent; (xiv) an amino acid or salt thereof; and (xv) an N-acetylamino acid or salt thereof. Solubility enhancers may increase the concentration of the drug or pharmaceutically acceptable salt thereof in the formulation. Useful solubility enhancers include, e.g., alcohols and polyalcohols.

An isotonizing agent may improve the tolerance of the formulation in certain instances. A common isotonizing agent is NaCl. Preferably, when the formulation is an isotonic intranasal dosage formulation, it includes about 0.9% NaCl (v/v) in the aqueous portion of the liquid carrier.

The thickeners may improve the overall viscosity of the composition, preferably to values close to those of the nasal mucosa. Suitable thickeners include methylcellulose, carboxymethylcellulose, polyvinypyrrolidone, sodium alginate, hydroxypropylmethylcellulose, and chitosan.

For topical compositions, a humectant or anti-irritant can improve the tolerability of the composition in repeated applications. Suitable compounds include, e.g. glycerol, tocopherol, mineral oils, and chitosan.

Various additional ingredients can be used in the compositions of the present invention. The compositions can comprise one or more further ingredients selected from a preservative, an antioxidant, an emulsifier, a surfactant or wetting agent, an emollient, a film-forming agent, or a viscosity modifying agent. These components can be employed and used at levels appropriate for the formulation based on the knowledge of one with ordinary skill in the pharmaceutical and formulation arts. The amounts could range from under 1 percent by weight to up to 90 percent or even over 99 percent by weight.

In one aspect, a preservative can be included. In another aspect, an antioxidant can be included. In another aspect, an emulsifier can be included. In another aspect, an emollient can be included. In another aspect, a viscosity modifying agent can be included. In another aspect, a surfactant or wetting agent can be included. In another aspect, a film forming agent can be included. In another aspect, the pharmaceutical composition is in the form selected from the group consisting of a gel, ointment, lotion, emulsion, cream, liquid, spray, suspension, jelly, foam, mousse, paste, tape, dispersion, aerosol. These components can be employed and used at levels appropriate for the formulation based on the knowledge of one with ordinary skill in the pharmaceutical and formulation arts.

In another aspect, the at least one preservative can be incorporated and selected from the group consisting of parabens (including butylparabens, ethylparabens, methylparabens, and propylparabens), acetone sodium bisulfite, alcohol, benzalkonium chloride, benzethonium chloride, benzoic acid, benzyl alcohol, boric acid, bronopol, butylated hydroxyanisole, butylene glycol, calcium acetate, calcium chloride, calcium lactate, cetrimide, cetylpyridinium chloride, chlorhexidine, chlorobutanol, chlorocresol, chloroxylenol, cresol, edetic acid, glycerin, hexetidine, imidurea, isopropyl alcohol, monothioglycerol, pentetic acid, phenol, phenoxyethanol, phenylethyl alcohol, phenylmercuric acetate, phenylmercuric borate, phenylmercuric nitrate, potassium benzoate, potassium metabisulfite, potassium nitrate, potassium sorbate, propionic acid, propyl gallate, propylene glycol, propylparaben sodium, sodium acetate, sodium benzoate, sodium borate, sodium lactate, sodium metabisulfite, sodium propionate, sodium sulfite, sorbic acid, sulfur dioxide, thimerosal, zinc oxide, and N-acetylcysteine, any other suitable preservative known in the art, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one antioxidant can be selected from the group consisting of acetone sodium bisulfite, alpha tocopherol, ascorbic acid, ascorbyl palmitate, butylated hydroxyanisole, butylated hydroxytoluene, citric acid monohydrate, dodecyl gallate, erythorbic acid, fumaric acid, malic acid, mannitol, sorbitol, monothioglycerol, octyl gallate, potassium metabisulfite, propionic acid, propyl gallate, sodium ascorbate, sodium formaldehyde sulfoxylate, sodium metabisulfite, sodium sulfite, sodium thiosulfate, sulfur dioxide, thymol, vitamin E polyethylene glycol succinate, and N-acetylcysteine, any other suitable antioxidant known in the art, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one emulsifier can be selected from the group consisting of acacia, agar, ammonium alginate, calcium alginate, carbomer, carboxymethylcellulose sodium, cetostearyl alcohol, cetyl alcohol, cholesterol, diethanolamine, glyceryl monooleate, glyceryl monostearate, hectorite, hydroxypropyl cellulose, hydroxypropyl starch, hypromellose, lanolin, lanolin alcohols, lauric acid, lecithin, linoleic acid, magnesium oxide, medium-chain triglycerides, methylcellulose, mineral oil, monoethanolamine, myristic acid, octyldodecanol, oleic acid, oleyl alcohol, palm oil, palmitic acid, pectin, phospholipids, poloxamer, polycarbophil, polyoxyethylene alkyl ethers, polyoxyethylene castor oil derivatives, polyoxyehtylene sorbitan fatty acid esters, polyoxyethylene stearates, polyoxyl 15 hydroxystearate, polyoxyglycerides, potassium alginate, propylene glycol alginate, propylene glycol dilaurate, propylene glycol monolaurate, saponite, sodium borate, sodium citrate dehydrate, sodium lactate, sodium lauryl sulfate, sodium stearate, sorbitan esters, starch, stearic acid, sucrose stearate, tragacanth, triethanolamine, tromethamine, vitamin E polyethylene glycol succinate, wax, and xanthan gum, any other suitable emulsifier known in the art, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one emollient can be selected from the group consisting of almond oil, aluminum monostearate, butyl stearate, canola oil, castor oil, cetostearyl alcohol, cetyl alcohol, cetyl palmitate, cholesterol, coconut oil, cyclomethicone, decyl oleate, diethyl sebacate, dimethicone, ethylene glycol stearates, glycerin, glyceryl monooleate, glyceryl monostearate, isopropyl isostearate, isopropyl myristate, isopropyl palmitate, lanolin, lanolin alcohols, lecithin, mineral oil, myristyl alcohol, octyldodecanol, oleyl alcohol, palm kernel oil, palm oil, petrolatum, polyoxyethylene sorbitan fatty acid esters, propylene glycol dilaurate, propylene glycol monolaurate, safflower oil, squalene, sunflower oil, tricaprylin, triolein, wax, xylitol, zinc acetate, any other suitable emollient known in the art or a combination thereof. These components can be employed and used at levels appropriate for the formulation based on the knowledge of one with ordinary skill in the pharmaceutical and formulation arts. The amounts could range from under 1 percent by weight to up to 60 percent by weight.

In another aspect, the at least one viscosity modifying agent can be selected from the group consisting of acacia, agar, alginic acid, aluminum monostearate, ammonium alginate, attapulgite, bentonite, calcium alginate, calcium lactate, carbomer, carboxymethylcellulose calcium, carboxymethylcellulose sodium, carrageenan, cellulose, ceratonia, ceresin, cetostearyl alcohol, cetyl palmitate, chitosan, colloidal silicon dioxide, corn syrup solids, cyclomethicone, ethylcellulose, gelatin, glyceryl behenate, guar gum, hectorite, hydrophobic colloidal silica, hydroxyethyl cellulose, hydroxyethylmethyl cellulose, hydroxypropyl cellulose, hydroxypropyl starch, hypromellose, magnesium aluminum silicate, maltodextrin, methylcellulose, myristyl alcohol, octyldodecanol, palm oil, pectin, polycarbophil, polydextrose, polyethylene oxide, polyoxyethylene alkyl ethers, polyvinyl alcohol, potassium alginate, propylene glycol alginate, pullulan, saponite, sodium alginate, starch, sucrose, sugar, sulfoburylether β-cyclodextrin, tragacanth, trehalose, and xanthan gum, any other suitable viscosity modifying agent known in the art, or a combination thereof. These components can be employed and used at levels appropriate for the formulation based on the knowledge of one with ordinary skill in the pharmaceutical and formulation arts. The amounts could range from under 1 percent by weight to up to 60 percent.

In another aspect, the at least one film forming agent can be selected from the group consisting of ammonium alginate, chitosan, colophony, copovidone, ethylene glycol and vinyl alcohol grafted copolymer, gelatin, hydroxypropyl cellulose, hypromellose, hypromellose acetate succinate, polymethacrylates, poly(methyl vinyl ether/maleic anhydride), polyvinyl acetate dispersion, polyvinyl acetate phthalate, polyvinyl alcohol, povidone, pullulan, pyroxylin, and shellac, any other suitable film-forming agent known in the art, or a combination thereof. These components can be employed and used at levels appropriate for the formulation based on the knowledge of one with ordinary skill in the pharmaceutical and formulation arts. The amounts could range from under 1 percent by weight to up to about 90 percent or even over 99 percent by weight.

In another aspect, the at least one surfactant or wetting agent can be selected from the group consisting of docusate sodium, phospholipids, sodium lauryl sulfate, benzalkonium chloride, cetrimide, cetylpyridinium chloride, alpha tocopherol, glyceryl monooleate, myristyl alcohol, poloxamer, polyoxyethylene alkyl ethers, polyoxyethylene castor oil derivatives, polyoxyethylene sorbitan fatty acid esters, polyoxyethylene stearates, polyoxyl 15 hydroxystearate, polyoxyglycerides, propylene glycol dilaurate, propylene glycol monolaurate, sorbitan esters, sucrose stearate, tricaprylin, and vitamin E polyethylene glycol succinate, any other suitable surfactant or wetting agent known in the art, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, a buffering agent can be included. In another aspect, an emollient can be included. In another aspect, an emulsifying agent can be included. In another aspect, an emulsion stabilizing agent can be included. In another aspect, a gelling agent can be included. In another aspect, a humectant can be included. In another aspect, an ointment base or oleaginous vehicle can be included. In another aspect, a suspending agent can be included. In another aspect an acidulant can be included. In another aspect, an alkalizing agent can be included. In another aspect, a bioadhesive material can be included. In another aspect, a colorant can be included. In another aspect, a microencapsulating agent can be included. In another aspect, a stiffening agent can be included. These components can be employed and used at levels appropriate for the formulation based on the knowledge of one with ordinary skill in the pharmaceutical and formulation arts. The amounts could range from under 1 percent by weight to up to 90 percent or even over 99 by weight.

One of ordinary skill in the pharmaceutical and formulation arts can determine the appropriate levels of the essential and optional components of the compositions of the present invention.

These formulations can be made using standard formulation and mixing techniques familiar to one of ordinary skill in the art of pharmaceuticals and formulations.

Methods of preparing the antipurinergic agent are also intended as part of the present invention and would be apparent to one of ordinary skill in the pharmaceutical and formulation arts using standard formulation and mixing techniques.

### Examples

The following examples further describe and demonstrate embodiments within the scope of the present invention. The Examples are given solely for purpose of illustration and are not to be construed as limitations of the present invention, as many variations thereof are possible without departing from the spirit and scope of the invention.

### Example 1: Composition for Intravenous Administration

The following composition is prepared using standard reconstitution techniques.

| Ingredient | Amount |
|---|---|
| Lyophilized antipurinergic | 10-200 mg/ml* |
| agent* | |

The lyophilized antipurinergic agent is diluted with sterile water for injection to obtain the desired aqueous solution for intravenous administration.

The compositions are useful for treating a nervous system disorder.

*An example of such a composition is the commercially available Germanin®, available from Bayer, which contains 1 g of lyophilized suramin per bottle for dilution with water.

### Example 2: Composition for Oral Administration

The following composition is prepared using standard mixing equipment and procedures.

| Ingredient | Amount |
|---|---|
| Antipurinergic agent | 10-200 mg/ml |
| Cremophore EL* (Sigma) | 10% weight |
| Water | QS to achieve |
| | the indicated |
| | levels of |
| | ingredients |

*Polyethoxylated castor oil, also known as Kolliphor EL.

The ingredients, and water are combined with mixing to form a homogeneous solution. The resultant solution can be packaged for oral administration.

The compositions are useful for treating a nervous system disorder.

### Example 3: Composition for Intranasal Delivery

The following composition is prepared using standard mixing equipment and procedures.

| Ingredient | Amount |
|---|---|
| Antipurinergic agent | 0-200 mg/ml |
| Methyl beta-cyclodextrin | 40% weight |
| (methyl betadex) | |
| Water | QS to achieve |
| | the indicated |
| | levels of |
| | ingredients |

The antipurinergic agent is dissolved in water with gentle mixing. The cyclodextrin is added with mixing until dissolved. The resultant solution is allowed to sit for 2 hours before using.

The composition can be packaged in a spray bottle for intranasal administration.

Alternatively, the compositions are prepared replacing the methyl Peta-cyclodextrin with an equal weight of caprylocaproyl macrogol-8 glycerides or and 2-(2-ethoxyethoxy)ethanol.

The compositions are useful for treating a nervous system disorder.

### Example 4 Three-Arm, Prospective, Randomized, Double Blind, Placebo—Controlled Trial Evaluating the Efficacy and Safety of 2 Doses of Suramin Vs. Placebo in Male Children with Autism Spectrum Disorder (ASD) Receiving Standard Treatment

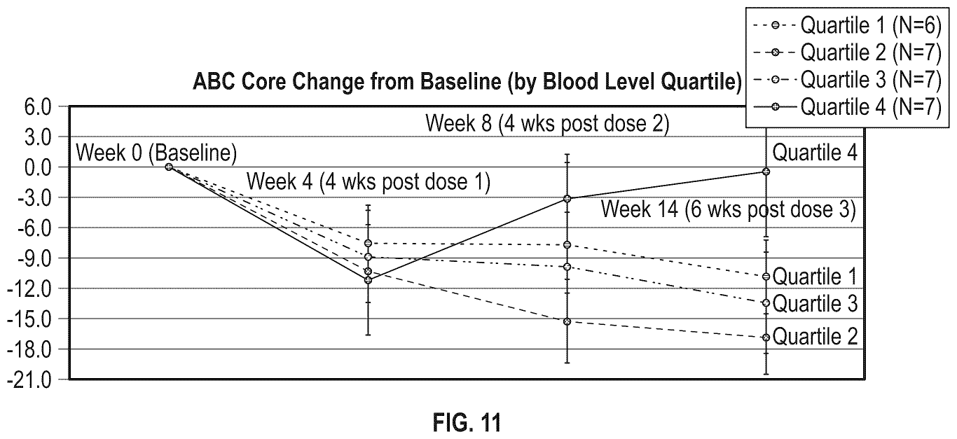

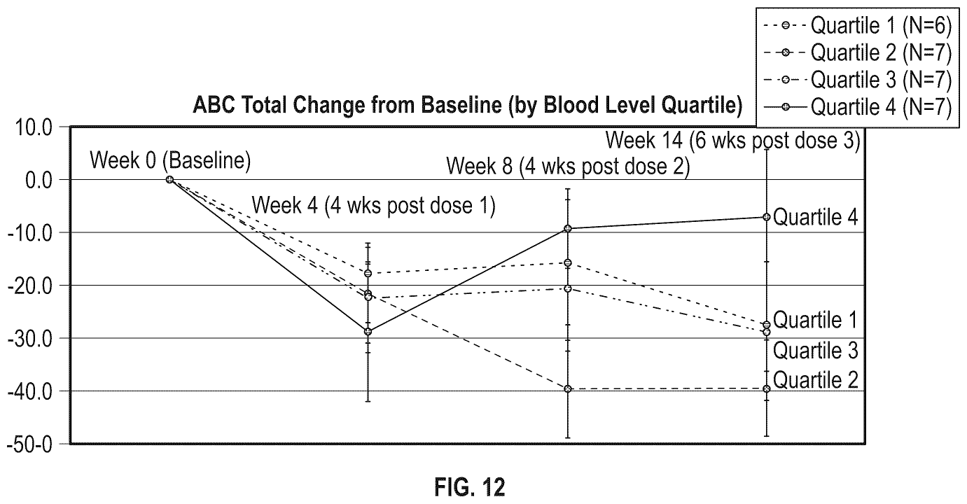

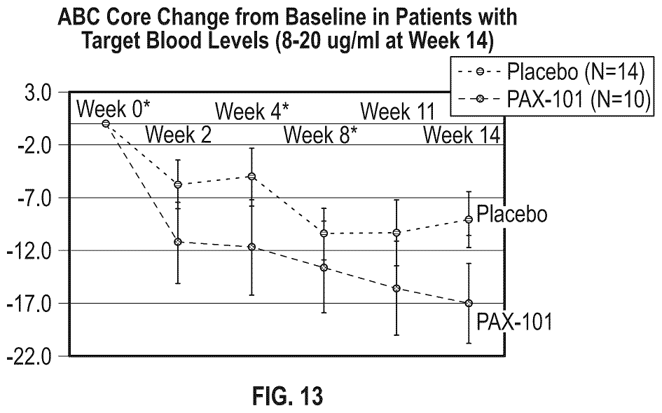

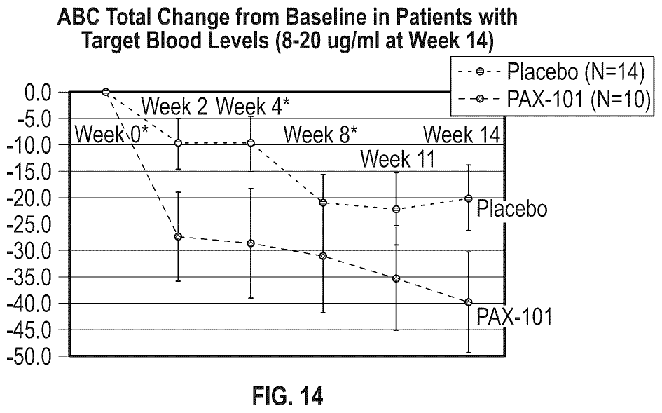

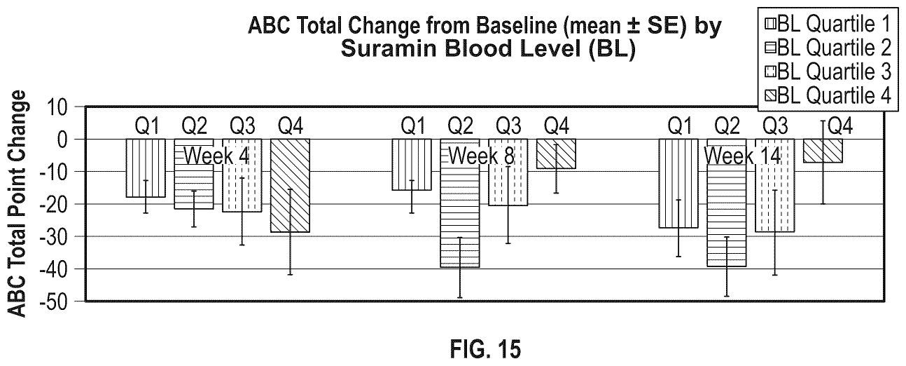

The results from this study demonstrate a dynamic nonlinear correlation between antipurinergic blood levels and efficacy as determined from the assessment of ASD using the Aberrant Behavior Checklist (ABC) and suramin blood levels. See, e.g. FIGS. 11-15. These results would not have been predicted based on previously disclosed dose-response linearity.

### SUMMARY

The bioanalytical data for this pharmacokinetic analysis came from a Three-Arm, Prospective, Randomized, Double Blind, Placebo—Controlled Trial evaluating the efficacy and safety of 2 doses of Suramin vs. Placebo in male children with Autism Spectrum Disorder (ASD) receiving standard treatment.

Approximately 52 study participants were enrolled and randomized to receive either suramin at 10 mg/kg (Arm A), or suramin at 20 mg/kg (Arm B), or placebo (Arm C) in a targeted 1:1:1 ratio, as per the randomization schedule and were matched by Age, ADOS (autism diagnosed observation schedule) and NVIQ (non-verbal IQ) per arm. This is the first time that multiple doses were studied in patients with neurodevelopmental disorders, and where they were observed over a long term duration. Previously, only a single 20 mg/kg dose had been described in an ASD study. See, R. K. Naviaux, “Antipurinergic therapy for autism—An in-depth review”, Mitochondrion 43, pp. 1-15 (2018), available online Dec. 16, 2017.

Each study participant attended a total of 7 visits, screening period (Visit 1.14 days), Randomization visit (Visit 2 day 1), treatment follow up visits (visit 3; day 14), treatment visit (visit 4; Day 28), treatment visit (visit 5; Day 56), End of treatment visit (visit 6; Day 77) and EOS (visit 7; day 98). Window period for screening until visit 2 will be ±14 days (thus the study procedures can be performed over 14 days) and for visit 2 onwards will be ±2 days (thus the procedures can be performed over 5 days) (Visit 2 to 7). The following Table 2 outlines the visit schedule:

TABLE 2
Visit #/Visit Type/Visit Day

| Visit 1 | Visit 2 | Visit 3 | Visit 4 | Visit 5 | Visit 6 | Visit 7 |
|---|---|---|---|---|---|---|
| Screening | Baseline | Treatment | Treatment | Treatment | Follow-up | End of study |
| Period (day −1 | D 1 +− 2 | D 14 ± 2 | (D 28 can be | D 56 can be | (D 77 can be | (D 98 can be |
| to day −14 | (can be | can be | done over 5 | done over 5 | done over 5 | done over 5 |
| can be done | done over | done over | days ± 2 | days ± 2) | days ± 2) | days ± 2) |
| over 14 days | 5 days | 5 days | | | | |

Plasma samples were collected for suramin pharmacokinetics (PK) analysis at visits 2, 4, 5, immediately before the infusion, and at 1 hour post-infusion and at visit 7. Suramin concentrations were measured by high-performance liquid chromatography coupled with a tandem mass spectrometer with Lower Limit of Quantitation (LLoQ) of 1 μg/mL.

The summary of mean pharmacokinetic parameters are summarized in Tables 3A-3D.

### Study Design and Sample Collection

The study design consists of parallel treatment arms, namely the three double blind treatment groups, either suramin at 10 mg/kg (Arm A), or, Suramin at 20 mg/kg (Arm B), or placebo (Arm C) randomized at ration 1;1;1.

See the study design in Table 4.

### Pharmacokinetic Parameters

A non-compartmental pharmacokinetic (PK) approach consistent with the intravenous route of administration was used to estimate PK parameters in Watson LIMS software (version 7.5). All parameters were generated from suramin individual concentrations in plasma from children on visits 1, 4, 5, and 7. All concentration values were in μg/mL, and all time points were in days as provided by the bioanalytical testing facility. Nominal dose concentrations and sampling times were used. The area under the plasma concentration-time curve (AUC) estimations were calculated on profiles having at least a quantifiable post-dose and pre-dose prior to the next dosing occasion. The observed maximum plasma concentration (Cₘₐₓ) and time of Cₘₐₓ (Tₘₐₓ) were determined directly from the data. Dose proportionality ratios for Cₘₐₓ, and AUC₀₋₉₆ days were calculated by dividing the pharmacokinetic parameter by the corresponding value in the lower dose groups and comparing with the corresponding fold change in dose.

### Pharmacokinetic Sample Concentrations

Control doses: Pre and post dose plasma concentrations in individual children were measured after 0 mg/kg dose of Suramin on Days 1, 28, 56, and 96 (visits 2, 4, 5, and 7) following intravenous route of administration. All measurements for this control were below the limit of quantitation (lower than 1 μg/mL).

All samples collected from control groups had a reported concentration below lowest level of quantitation (BLQ). For other groups, the sample collected at time point zero at the start of the study were all below lowest level of quantitation. Other time points had quantifiable concentrations for all samples that were submitted.

Table 5 shows pre and post Dose Plasma Concentrations in Individual Children after 10 mg/kg Dose of Suramin on Days 1, 28, 56, and 96 (visits 2, 4, 5, and 7) Following Intravenous Route of Administration.

Table 6 shows pre and post Dose Plasma Concentrations in Individual Children after 20 mg/kg Dose of Suramin on Days 1, 28, 56, and 96 (visits 2, 4, 5, and 7) Following Intravenous Route of Administration.

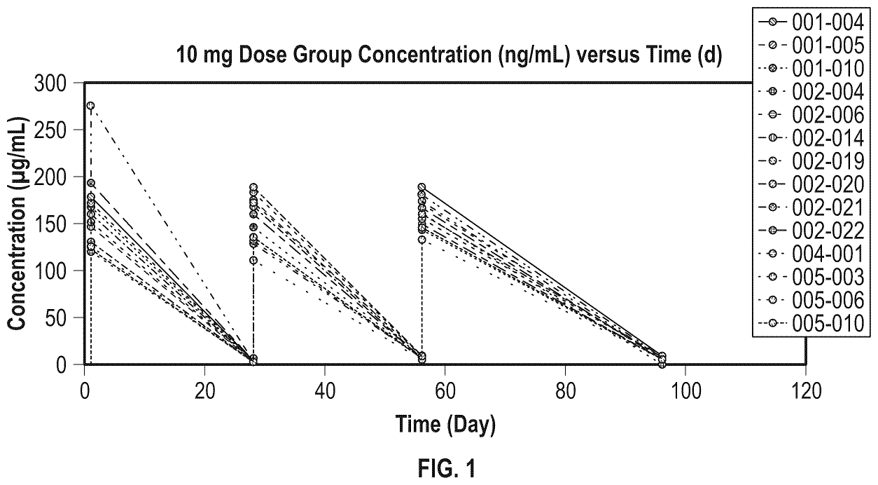

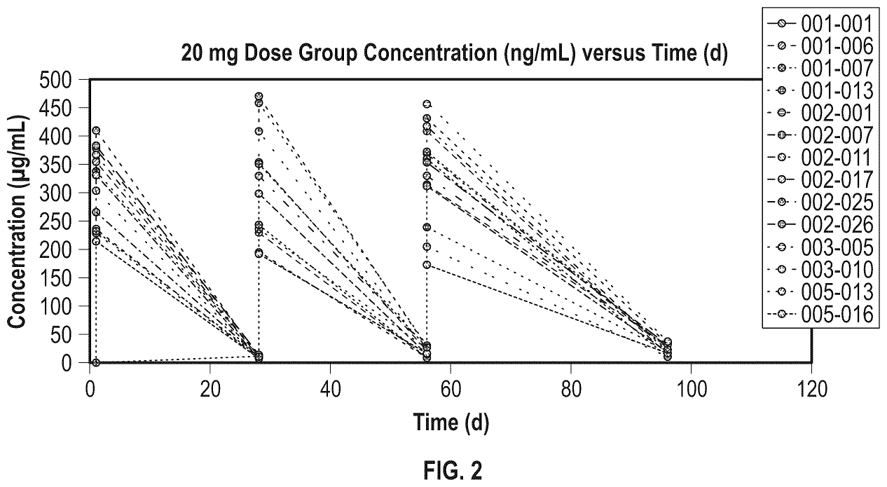

A general agreement was observed between dose levels and measured plasma concentrations. See FIG. 1 and FIG. 2 These figures show that with increasing dose levels, higher plasma concentrations were measured.

### Pharmacokinetic Analysis

Pharmacokinetic parameters were estimated using a non-compartmental approach consistent with intravenous infusion route of administration. Suramin individual concentrations in plasma and nominal times were used.

See Table 7.

For area under the curve (AUC) estimation, measured plasma concentration for post dose and pre dose before the next dosing event were needed. For this study, samples for pharmacokinetic analysis were collected on days 1, 28, 56, and 96.

Whenever possible, standard deviation and coefficient of variation were determined by Watson LIMS software. For analyses that were not supported by Watson (i.e. custom comparisons), Excel was used and the statistics reported as determined by excel algorithms. Note that in either case, when the number of data points was less than three, standard deviation and coefficient of variation could not be calculated.

### Mean Pharmacokinetic Parameters

Pharmacokinetics parameters are summarized in Tables 3A-3D for the two dose levels, 10 and 20 mg/kg. The route of administration was intravenous infusion over a 30 minute period on visits 2, 4, and 5.

Tₘₐₓ has a median value of 28 days and ranges from 1 to 56 days for both dose groups 10 and 20 mg/kg and occurred 1 hour after the infusion on those dates.

Systemic exposure, as assessed by suramin Cₘₐₓ, and AUC₀₋₉₆ increased with the increase in dose levels 10, and 20 mg/kg. The increases in Cₘₐₓ were less than dose proportional from 10 mg/kg to 20 mg/kg dose range, whereas AUC₀₋₉₆ day values were dose proportional.

Mean values are shown in Tables 3A-3D.

Maximum plasma concentration, as assessed by Cₘₐₓ, was shown to be less than dose proportional, whereas systemic exposure, as assessed by AUC₀₋₉₆ was shown to be nearly dose proportional. Since only predose, 1 hour post dose, and before the next dose administration samples were collected, it was not possible to determine the exact distribution and elimination curves and therefore extrapolated parameters such as AUC_{0-∞} and T_{1/2} were not determinable. Note that the 10 mg dose group did not show significant accumulation which may be due to a half-life shorter than 28 days for this low dose level. The fact that 20 mg dose group did show accumulation implies that the half-life is greater than 28 days for the high dose level and possibly due to limitations in renal clearance. The data also showed that adverse effects in the suramin treated patients, such as visible rash or vomiting, occurred mostly in the 20 mg/kg dose group and only one event in the lower dose 10 mg/kg group. The minimum observed AUC in 20 mg/kg dose group was higher than the maximum AUC value in 10 mg/kg dose group. Tₘₐₓ, Cₘₐₓ, and AUC were observed to have a weak to no correlation with age and body mass index (BMI). Suramin accumulation was also observed in the high dose group 20 mg/kg. A greater change in ABC scores from baseline was observed with lower exposures.

Tables 3A-3D: Summary of Mean Pharmacokinetic Parameters of Suramin in Children after 10 or 20 mg/kg Dose of Suramin on Days 1, 28, 56, and 96 (visits 4, 5, and 7) Following Intravenous Route of Administration

TABLE 3A
Plasma Suramin Treatment PUR-ONQ-ASD-001

| | Parameters | | | | | | | |
|---|---|---|---|---|---|---|---|---|
| | | | | | | | AUCᵢₙₜₑᵣᵥₐₗ/ | |
| | | Dose | | | AUC_{Interval} | Cₘₐₓ/Dose | Dose | |
| | Tₘₐₓ | Interval | Cₘₐₓ | Cₘᵢₙ | (μg*Days/ | (μg/mL/ | (μg*Days/ | Dose |
| 10 mg | (d) | (d) | (μg/mL) | (μg/mL) | mL) | mg) | mL/mg) | (mg) |
| Mean | 28.3 | NA | 244 | 4.3 | 4780 | 24.4 | 478 | 10.0 |
| SD | 23.2 | NA | 252 | 1.02 | 1550 | 25.2 | 155 | NA |
| % CV | 82.0 | NA | 103 | 23.8 | 32.4 | 103 | 32.4 | NA |

TABLE 3B
Ratio of Parameters to 10 mg Dose

| | Cₘₐₓ | AUC_{Interval} | Cₘₐₓ/Dose | AUCᵢₙₜₑᵣᵥₐₗ/Dose |
|---|---|---|---|---|
| | (μg/mL) | (μg*Days/mL) | (μg/mL/mg) | (μg*Days/mL/mg) |
| Mean | NA | NA | NA | NA |
| SD | NA | NA | NA | NA |
| % CV | NA | NA | NA | NA |

TABLE 3C
Plasma Suramin Treatment PUR-ONQ-ASD-001

| | Parameters | | | | | | | |
|---|---|---|---|---|---|---|---|---|
| | | | | | | | AUCᵢₙₜₑᵣᵥₐₗ/ | |
| | | Dose | | | AUC_{Interval} | Cₘₐₓ/Dose | Dose | |
| | Tₘₐₓ | Interval | Cₘₐₓ | Cₘᵢₙ | (μg*Days/ | (μg/mL/ | (μg*Days/ | Dose |
| 20 mg | (d) | (d) | (μg/mL) | (μg/mL) | mL) | mg) | mL/mg) | (mg) |
| Mean | 28.4 | NA | 365 | 10.6 | 9510 | 18.3 | 475 | 20.0 |
| SD | 24.6 | NA | 72.1 | 2.24 | 2890 | 3.61 | 144 | NA |
| % CV | 86.7 | NA | 19.7 | 21.1 | 30.4 | 19.7 | 30.4 | NA |

TABLE 3D
Ratio of Parameters to 10 mg Dose

| | Cₘₐₓ | AUC_{Interval} | Cₘₐₓ/Dose | AUCᵢₙₜₑᵣᵥₐₗ/Dose |
|---|---|---|---|---|
| | (μg/mL) | (μg*Days/mL) | (μg/mL/mg) | (μg*Days/mL/mg) |
| Mean | 1.49 | 1.99 | 0.75 | 0.99 |
| SD | 0.29 | 1.86 | 0.14 | 0.93 |
| % CV | 0.19 | 0.94 | 0.19 | 0.94 |

### Table 4: Pharmacokinetic Subset Experimental Study Design and Sample Collection

TABLE 4

| | Visit 1 | Visit 2 | Visit 3 | Visit 4 |
|---|---|---|---|---|
| PK Sampling^{a} | Screening | Baseline | Treatment | Treatment |
| | | x^{a} | | x^{a} |

| Visit 5 | Visit 6 | Visit 7 |
|---|---|---|
| Treatment | Follow-up | End of Study |
| x^{a} | | x |

^{a}PK samples were collected immediately before the infusion, and at 1 h post-infusion

Table 5: Pre and Post Dose Plasma Concentrations in Individual Children after 10 mg/kg Dose of Suramin on Days 1, 28, 56, and 96 (visits 2, 4, 5, and 7) Following Intravenous Route of Administration

TABLE 5

| | Plasma Concentration (μg/mL) | | | | | | |
|---|---|---|---|---|---|---|---|
| VISIT | 2 | 2 | 4 | 4 | 5 | 5 | 7 |
| SAMPLETIME | Pre- | 1 | Pre- | 1 | Pre- | 1 | EOS |
| (HR) | dose | | dose | | dose | | |
| Day | 1 | 1 | 28 | 28 | 56 | 56 | 96 |
| N | 15 | 15 | 15 | 15 | 15 | 15 | 14 |
| Mean | NA | 229 | 4.31 | 157 | 7.96 | 164 | 7.87 |
| SD | NA | 258 | 1.02 | 24.1 | 1.83 | 18.6 | 1.49 |
| % CV | NA | 113 | 23.7 | 15.4 | 23.0 | 11.3 | 18.9 |

Table 6: Pre and Post Dose Plasma Concentrations in Individual Children after 20 mg/kg Dose of Suramin on Days 1, 28, 56, and 96 (visits 2, 4, 5, and 7) Following Intravenous Route of Administration

TABLE 6

| | Plasma Concentration (μg/mL) | | | | | | |
|---|---|---|---|---|---|---|---|
| VISIT | 2 | 2 | 4 | 4 | 5 | 5 | 7 |
| SAMPLETIME | Pre- | 1 | Pre- | 1 | Pre- | 1 | EOS |
| (HR) | dose | | dose | | dose | | |
| Day | 1 | 1 | 28 | 28 | 56 | 56 | 96 |
| N | 19 | 18 | 16 | 16 | 15 | 15 | 15 |
| Mean | NA | 328 | 11 | 327 | 21.1 | 332 | 23.4 |
| SD | NA | 67.3 | 2.08 | 91.5 | 7.55 | 85.7 | 8.21 |
| % CV | NA | 20.5 | 18.9 | 28 | 35.8 | 25.8 | 35.1 |

Table 7. Average pharmacokinetic parameters (with standard deviation, S.D.) for children after 10 mg or 20 mg/kg of Suramin on days 1, 28, 56, and 96 (visits 4, 5, and 7) following intravenous route of administration.

TABLE 7

| | | 10 mg/kg Dose | | 20 mg/kg Dose | |
|---|---|---|---|---|---|
| | | n = 13 | | n = 14 | |
| Parameter | Units | Mean | S.D. | Mean | S.D. |
| Tₘₐₓ | (d) | 28.3 | 22.5 | 26.4 | 25.2 |
| C_{max, Oveall} | (μg/mL) | 180.6 | 33.3 | 358.6 | 72.3 |
| C_{min. overall} | (μg/mL) | 4.4 | 1.0 | 10.6 | 2.3 |
| C_{min, Visit 4} | (μg/mL) | 4.4 | 1.0 | 10.9 | 2.2 |
| C_{min, Visit 5} | (μg/mL) | 7.7 | 1.7 | 21.9 | 7.1 |
| C_{min, Visit 7} | (μg/mL) | 7.7 | 1.4 | 24.2 | 7.9 |
| AUC_{Overall} | (μg*Days/mL) | 4600.0 | 413.9 | 10203.6 | 2269.3 |
| AUC_{Visit 4} | (μg*Days/mL) | 1195.6 | 209.4 | 2431.4 | 467.7 |
| AUC_{Visit 5} | (μg*Days/mL) | 1386.7 | 198.6 | 3121.4 | 838.5 |
| AUC_{Visit 7} | (μg*Days/mL) | 2017.7 | 201.5 | 4650.7 | 1194.4 |
| Cₘₐₓ/Dose | (μg/mL/mg) | 18.1 | 3.3 | 18.0 | 3.6 |
| AUCᵢₙₜₑᵣᵥₐₗ/Dose | (μg*Days/mL/mg) | 460.0 | 41.4 | 510.1 | 113.7 |

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the methods and compositions of the present invention, where the term comprises is used with respect to the recited steps of the methods or components of the compositions, it is also contemplated that the methods and compositions consist essentially of, or consist of, the recited steps or components. Furthermore, the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

Furthermore, it should be recognized that in certain instances a composition can be described as composed of the components prior to mixing, because upon mixing certain components can further react or be transformed into additional materials.

All percentages and ratios used herein, unless otherwise indicated, are by weight. It is recognized the mass of an object is often referred to as its weight in everyday usage and for most common scientific purposes, but that mass technically refers to the amount of matter of an object, whereas weight refers to the force experienced by an object due to gravity. Also, in common usage the “weight” (mass) of an object is what one determines when one “weighs” (masses) an object on a scale or balance.

What is claimed is:

1. A method of treating a nervous system disorder in a mammal in need thereof, comprising administering to said mammal a pharmaceutical composition comprising an effective amount of an antipurinergic agent, or a pharmaceutically acceptable salt, ester, solvate, or prodrug thereof, according to a dosing regimen comprising (a) an optional loading dosing regimen and (b) a subsequent maintenance dosing regimen, wherein
(a) said optional loading dosing regimen is selected from (i) a single loading dose administered once, or (ii) multiple loading doses each administered with a frequency ranging from about once daily to about once every third month, wherein each loading dose comprises from about 3 mg/kg to about 30 mg/kg of the antipurinergic agent, and
(b) said subsequent maintenance dosing regimen is selected from multiple maintenance doses each administered with a frequency ranging from about three times daily to about once every third month, wherein each maintenance dose comprises from about 1 mg/kg to about 15 mg/kg of the antipurinergic agent.

2. The method according to claim 1 wherein said multiple loading doses of (a) (ii) are each administered with a frequency selected from once daily, four times per week, three times per week, two times per week, once per week, three times per month, two times per month, once per month, once every other month, or once every third month, wherein each loading dose comprises from about 3 mg/kg to about 30 mg/kg of the antipurinergic agent, and wherein said multiple maintenance doses of (b) are each administered with a frequency selected from the group consisting of three times daily, twice daily, once daily, four times per week, three times per week, two times per week, once per week, three times per month, two times per month, once per month, once every other month, or once every third month, wherein each loading dose comprises from about 1 mg/kg to about 15 mg/kg of the antipurinergic agent.

3. The method according to claim 1, wherein the molar ratio of the antipurinergic agent in each individual loading dose to the antipurinergic agent in each maintenance dose is from about 1:1.25 to about 4:1.

4. The method according to claim 1 further comprising a regimen wherein the loading dose or doses of from 3 mg/kg to about 30 mg/kg, which is defined as an initial loading dose or doses, is stepped down to one or more lower intermediate loading doses, prior to commencement of the administration of the maintenance doses.

5. The method according to claim 1 including a loading dosage regimen, wherein:
(i) said loading dosage regimen is administered to obtain a Cmin plasma level of about 8 μg/ml to 24 μg/ml of the antipurinergic agent and wherein the maintenance dosing regimen is continued to maintain a Cmin plasma level of about 4 μg/ml to about 18 μg/ml of the antipurinergic agent;
(ii) said loading dosage regimen is administered to obtain a Cₘₐₓ plasma level of about 100 μg/ml to about 500 μg/ml, or about 150 μg/ml to about 450 μg/ml, or about 200 μg/ml to about 350 μg/ml of the antipurinergic agent and wherein the maintenance dosing regimen is continued to maintain a Cₘₐₓ plasma level of about 50 μg/ml to about 300 μg/ml, or about 100 μg/ml to about 200 μg/ml, or about 125 μg/ml to about 175 μg/ml of the antipurinergic agent; or
(iii) said loading dosage regimen is administered to obtain an AUC for the plasma level for the antipurinergic agent of about 1500 to about 7000 μg*day/L, or about 1700 to about 6500 μg*day/L, or about 2000 to about 6000 μg*day/L and wherein the maintenance dosing regimen is continued until an AUC for the plasma level for the antipurinergic agent of about 700 to about 3000 μg*day/L, or about 900 about 2000 μg*day/L, or about 1200 to about 1500 μg*day/L is attained.

6. The method according to claim 1 wherein the mean plasma level of the antipurinergic agent attained in the maintenance dosing regimen is about 20% to about 80% of the mean plasma level of the antipurinergic agent attained in the loading dosing regimen or wherein the mean plasma level of the antipurinergic agent attained in the loading dosing regimen is about 125% to about 400% of the mean plasma level of the antipurinergic agent attained in the maintenance dosing regimen.

7. The method according to claim 1 wherein the Cmin plasma level of the antipurinergic agent attained in the maintenance dosing regimen is about 20% to about 80% of the Cmin plasma level of the antipurinergic agent attained in the loading dosing regimen or wherein the Cmin plasma level of the antipurinergic agent attained in the loading dosing regimen is about 125% to about 400% of the Cmin plasma level of the antipurinergic agent attained in the maintenance dosing regimen.

8. The method according to claim 1 wherein the Cₘₐₓ plasma level of the antipurinergic agent attained in the maintenance dosing regimen is about 20% to about 80% of the Cₘₐₓ plasma level of the antipurinergic agent attained in the loading dosing regimen or wherein the Cₘₐₓ plasma level of the antipurinergic agent attained in the loading dosing regimen is about 125% to about 400% of the Cₘₐₓ plasma level of the antipurinergic agent attained in the maintenance dosing regimen.

9. The method according to claim 1 wherein the AUC of the antipurinergic agent attained in the maintenance dosing regimen is about 20% to about 80% of the AUC of the antipurinergic agent attained in the loading dosing regimen or wherein the AUC of the antipurinergic agent attained in the loading dosing regimen is about 125% to about 400% of the AUC of the antipurinergic agent attained in the maintenance dosing regimen.

10. The method according to claim 1 wherein at least one of the following PK parameters is achieved for the optional loading dose, selected from the group consisting of a Cmin of about 8 μg/ml to about 24 μg/ml, a Cₘₐₓ of about 100 μg/ml to about 500 μg/ml, or an AUC of about 1500 to about 7000 μg*day/L or wherein at least one of the following PK parameters is achieved for the maintenance dose, selected from the group consisting of a Cmin of about 4 μg/ml to about 18 μg/ml, a Cₘₐₓ of about 50 μg/ml to about 300 μg/ml, or an AUC of about 700 to about 3000 μg*day/L.

11. The method according to claim 1 wherein the mammal is a human.

12. The method according to claim 11 wherein the method is used to adjust the loading and maintenance doses according to efficacy and/or safety/tolerability endpoints selected from:
a) anxiety or anxiety-like behavior,
b) willingness to explore the environment,
c) social interaction,
d) spatial learning and memory,
e) learning and memory,
f) irritability, agitation and or crying,
g) lethargy and/or social withdrawal,
h) stereotypic behavior,
i) hyperactivity and/or noncompliance, and
j) restrictive and/or repetitive behaviors.

13. The method according to claim 11 wherein said nervous system, disorder is selected from autism spectrum disorder (ASD), fragile X syndrome (FXS), fragile X-associated tremor/ataxia syndrome (FXTAS), myalgic encephalomyelitis/chronic fatigue syndrome (ME/CFS), post-traumatic stress syndrome (PTSD), Tourette's syndrome (TS), Parkinson's disease (PD), Angelman syndrome (AS), chronic Lyme disease and other nervous system disorders associated with tick-borne illnesses, and nervous system and central nervous system (CNS) disorders associated with viral infections, including their long term effects.

14. The method according to claim 1 wherein said antipurinergic agent is selected from berberine, emodin, suramin, tangeretin, A-438079, A-839977, A-804598, JNJ-47965567, and KN-62, pharmaceutically acceptable salts, esters, prodrugs, and solvates thereof, and combinations thereof.

15. The method according to claim 1 wherein said antipurinergic agent is suramin, or a pharmaceutically acceptable salt, ester, solvate, or prodrug thereof.

16. The method according to claim 15 wherein the pharmaceutically acceptable salt is selected from an alkali metal salt, an alkaline earth metal salt, and an ammonium salt.

17. The method according to claim 16 wherein said salt is a sodium salt.

18. The method according to claim 17 wherein said salt is the hexa-sodium salt.

19. The method according to claim 1 wherein said composition is administered nasally or intranasally (IN).

20. The method according to claim 1 wherein said composition is administered intravenously (IV).

21. A kit for treating a nervous system, psychiatric, or neurologic disorder in a mammal in need thereof, comprising first and second components for administering a pharmaceutical composition comprising an effective amount of an antipurinergic agent, or a pharmaceutically acceptable salt, ester, solvate, or prodrug thereof, and, optionally, instructions for administering the pharmaceutical composition, wherein
(a) the first component includes administering the composition according to a loading dosing regimen selected from (i) a single loading dose administered once, or (ii) multiple loading doses each administered with a frequency ranging from about once daily to about once every third month, wherein each loading dose comprises from about 3 mg/kg to about 30 mg/kg of the antipurinergic agent, and
(b) the second component includes administering the composition according to a subsequent maintenance dosing regimen selected from multiple maintenance doses each administered with a frequency ranging from about three times daily to about once every third month, wherein each maintenance dose comprises from about 1 mg/kg to about 15 mg/kg of the antipurinergic agent.

22. The kit according to claim 21 wherein said antipurinergic agent or a pharmaceutically acceptable salt, ester, solvate, or prodrug thereof is suramin, or a pharmaceutically acceptable salt, ester, solvate, or prodrug thereof.

23. A method of inhibiting or modulating a purinergic receptor in a mammal in need thereof, comprising administering to said mammal a pharmaceutical composition comprising an effective amount of an antipurinergic agent, or a pharmaceutically acceptable salt, ester, solvate, or prodrug thereof, according to a dosing regimen comprising (a) a loading dosing regimen and (b) a subsequent maintenance dosing regimen, wherein
(a) said loading dosing regimen is selected from (i) a single loading dose administered once, or (ii) multiple loading doses each administered with a frequency ranging from about once daily to about once every third month, wherein each loading dose comprises from about 3 mg/kg to about 30 mg/kg of the antipurinergic agent, and
(b) said subsequent maintenance dosing regimen is selected from multiple maintenance doses each administered with a frequency ranging from about three times daily to about once every third month, wherein each maintenance dose comprises from about 1 mg/kg to about 15 mg/kg of the antipurinergic agent.

24. The method according to claim 23 wherein said antipurinergic agent or a pharmaceutically acceptable salt, ester, solvate, or prodrug thereof is suramin, or a pharmaceutically acceptable salt, ester, solvate, or prodrug thereof.

25. A pharmaceutical composition comprising an effective amount of an antipurinergic agent, or a pharmaceutically acceptable salt, ester, solvate, or prodrug thereof for use in a method for treating a nervous system disorder in a mammal in need thereof, wherein the composition is administered according to a dosing regimen comprising (a) a loading dosing regimen and (b) a subsequent maintenance dosing regimen, wherein
(a) said loading dosing regimen is selected from (i) a single loading dose administered once, or (ii) multiple loading doses each administered with a frequency ranging from about once daily to about once every third month, wherein each loading dose comprises from about 3 mg/kg to about 30 mg/kg of the antipurinergic agent, and
(b) said subsequent maintenance dosing regimen is selected from multiple maintenance doses each administered with a frequency ranging from about three times daily to about once every third month, wherein each maintenance dose comprises from about 1 mg/kg to about 15 mg/kg of the antipurinergic agent.

26. The method according to claim 25 wherein said antipurinergic agent or a pharmaceutically acceptable salt, ester, solvate, or prodrug thereof is suramin, or a pharmaceutically acceptable salt, ester, solvate, or prodrug thereof.
